(19) United States
(12) Patent Application Publication
(10) Pub. No.: US 2008/0015189 A1
(43) Pub. Date: Jan. 17, 2008

(54) Antimicrobial photoinactivation using chalcogen analogs of benzo(A)phenoxazinium dyes

(75) Inventors: Michael R. Hamblin, Revere, MA (US); James W. Foley, Andover, MA (US)

Correspondence Address: EDWARDS ANGELL PALMER & DODGE LLP, P.O. BOX 55874, BOSTON, MA, 02205, US

(73) Assignee: The General Hospital Corporation, Boston, MA (US)

(21) Appl. No.: 11/820,362

(22) Filed: Jun. 19, 2007

Related U.S. Application Data
(60) Provisional application No. 60/815,226, filed on Jun. 20, 2006.

(51) Int. Cl. A61K 31/5415; A61K 31/33; A61P 31/00; C07D 293/10; C07D 513/22; C07C 391/00; A61K 31/536

(52) U.S. Cl. 514/224.8; 514/183; 514/229.5; 544/1; 544/31; 562/899; 564/440

(57) ABSTRACT

Compositions and methods for the use of photoactivatable antimicrobial chalcogen analogs of benzophenoxazinium dyes in the treatment of infections are provided.

### RELATED APPLICATIONS/PATENTS & INCORPORATION BY REFERENCE

The application claims the benefit of U.S. provisional application Ser. No. 60/815,226, filed Jun. 20, 2006, the entire disclosure of which is incorporated herein by reference. Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF GOVERNMENT SUPPORT

The work leading to the present invention was funded in part by grant number R01AI050875 from the National Institute of Allergy and Infectious Disease of the National Institutes of Health. Accordingly, the United States Government may have certain rights to this invention.

### BACKGROUND OF THE INVENTION

Photodynamic therapy is a medical treatment that uses the energy of actinic light to selectively inactivate diseased tissues and infectious pathogens.¹ The therapy involves the systemic or topical administration of a target-localizing, non-toxic dye or photosensitizer that becomes, or in reaction with molecular oxygen, generates, a cytotoxin when illuminated with light of the appropriate wavelength.^{2,3} Since the power and safety of the method derive from its dual selectivity—inactivation occurs only where the photosensitizer and light are simultaneously present—effective treatment relies on the photosensitizer having a high degree of specificity and affinity for selected biological targets.⁴

Because of the well-known increase in multi-antibiotic resistance amongst pathogenic microbes of all classes, photodynamic therapy has attracted growing attention as a possible treatment for localized infections.¹⁹ Preclinical studies have found that many commonly used porphyrin photosensitizers are effective at inactivating Gram-positive²⁰ bacteria but are considerably less efficacious against Gram-negative organisms²¹; fungal cells, such as Candida, are generally midway in susceptibility between both types of bacteria.²² Cationic photosensitizers, on the other hand, have been found to be effective against both Gram-positive and Gram-negative bacteria²³, which is consistent with the observation that most dyes used in pathology to identify bacteria and bacterial spores bear a delocalized cationic charge, as exemplified by the Gram stain/counter-stain pair, Gentian Violet and Fuchsin²⁴, and the Wirtz-Conklin pair, Malachite Green and Safranine-O.²⁵ For example, Methylene Blue, Toluidine Blue and Dimethylmethylene Blue, phenothiazinium dyes closely related to EtNBS (2), are safe and effective antimicrobial photosensitizers²⁶ that are currently the only photodynamic agents being used for clinical antimicrobial applications in the oral cavity for such indications as periodontitis²⁷ and tooth cavity sterilization.²⁸

Nevertheless, the use of these agents suffers constraints that might impair their activities. Firstly, they must be used at relatively high concentrations to achieve multiple logs of cell killing with reasonable light fluences. Secondly, they are much more effective when the agent remains present in the incubation mixture during illumination, compared to when the microbes are washed free from unbound agent before light delivery.²² Thus, improved photodynamic agents would be beneficial in the development of effective anti-microbial treatments.

### SUMMARY OF THE INVENTION

The present invention is directed to photodynamic agents of the benzophenoxazinium family of cationic chromophores and use of the same as anti-microbial agents. In particular, three chalcogen (O, S, Se)-substituted benzo[a]phenoxazinium analogs are described. Synthesis of the agents is further described.

In one aspect, the present invention provides a method for decreasing the activity of an unwanted organism in a subject, said method comprising the steps of:

i) providing an effective amount of a photoactive chalcogen analog of a benzophenoxazinium dye to a pathogen; and

ii) light-activating the chalcogen analog to produce a phototoxic species, thereby decreasing the activity of the unwanted organism in the subject.

In various embodiments, the chalcogen analog is a selenium chalcogen analog, sulfur chalcogen analog, and oxygen chalcogen analog. The chalcogen analog can further comprises a targeting moiety.

In one embodiment, the selenium chalcogen analog is 5-ethylamino-9-diethylaminobenzo[a]phenoselenazinium chloride.

In another embodiment, the sulfur chalcogen analog is 5-ethylamino-9-diethylaminobenzo[a]phenothiazinium chloride.

In yet another embodiment, the oxygen chalcogen analog is 5-ethylamino-9-diethylaminobenzo[a]phenoxazinium chloride.

In various embodiments, the unwanted organism is a virus, bacteria (e.g., Enterococcus faecalis, Escherichia coli) or fungi (e.g., Candida albicans). Methods of the invention can further comprise the step of obtaining the chalcogen analog.

In another aspect, the invention provides a kit for decreasing the activity of an unwanted organism in a subject, the kit comprising a chalcogen analog of a benzophenoxazinium dye and instructions for using the chalcogen analog in accordance with the methods of the invention.

In yet another aspect, the invention provides compounds such as chalcogen analogs. The compounds can be represented by the following structure (Formula I): 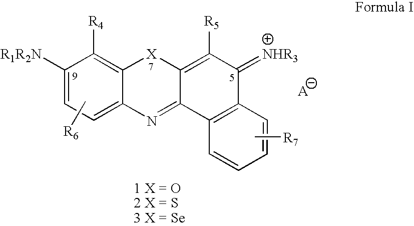
in which

X is selected from the group consisting of O, S, and Se;

R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure;

R₄ and R₅ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and R₆, together with the N atom to which R₂ is attached and two or more atoms in the ring to which R₆ is attached, can form an optionally substituted heterocyclic ring having from 5 to 8 atoms in the heterocyclic ring structure; or R₃ and R₅, together with the N atom to which R₃ is attached and two atoms in the ring to which R₅ is attached, can form an optionally substituted heterocyclic ring having from 5 to 8 atoms in the heterocyclic ring structure; or R₃ and R₇, together with the N atom to which R₃ is attached and two or more atoms in the ring system to which R₇ is attached, can form an optionally substituted heterocyclic ring having from 5 to 8 atoms in the heterocyclic ring structure;

R₆ and R₇ each independently represents 0-2 groups selected from the group consisting of C₁-C₆ alkyl, C₁-C₆ alkoxy, halogen, cyano, or nitro; and

A is an anion.

In certain preferred embodiments, in a compound of Formula I, X is S or Se. In certain preferred embodiments of Formula I, R₁, R₂ and R₃ are each independently C₁-C₆ alkyl; in more preferred embodiments, each of R₁, R₂ and R₃ is ethyl. In certain preferred embodiments of Formula I, R₄ and R₅ are each independently H, C₁-C₆ alkyl or C₁-C₆ alkoxy; in more preferred embodiments, R₄ and R₅ are each H. In certain preferred embodiments, when R₄ is a group other than H, then at least one of R₁ and R₂ is H, to decrease potential steric interactions. In certain preferred embodiments, when R₅ is a group other than H, then R₃ is H, to decrease potential steric interactions. In certain preferred embodiments, R₆ and R₇ are absent. In certain preferred embodiments, A is a monovalent or divalent anion, more preferably a monovalent anion, more preferably an anion selected from the group consisting of fluoride, chloride, bromide, iodide, tosylate (p-toluenesulfonate), mesylate (methylsulfonate), triflate (trifluoromethylsulfonate), acetate, trifluoroacetate, and benzoate.

In one embodiment, the invention provides a method for preparing a compound represented by Formula Ib, 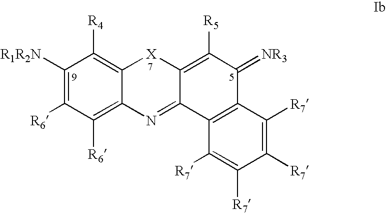
in which X is S or Se; R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure; R₄ and R₅ are each independently H, C₁-C₆ alkyl, cycloalkyl, aryl, C₁-C₆ alkoxy, halogen, cyano, or nitroor R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and R₆, together with the N atom to which R₂ is attached and two or more atoms in the ring to which R₆ is attached, can form an optionally substituted heterocyclic ring having from 5 to 8 atoms in the heterocyclic ring structure; or R₃ and R₅, together with the N atom to which R₃ is attached and two atoms in the ring to which R₅ is attached, can form an optionally substituted heterocyclic ring having from 5 to 8 atoms in the heterocyclic ring structure; or R₃ and R₇, together with the N atom to which R₃ is attached and two or more atoms in the ring system to which R₇ is attached, can form an optionally substituted heterocyclic ring having from 5 to 8 atoms in the heterocyclic ring structure; and R₆′ and R′₇ are, independently for each occurrence, H, C₁-C₆ alkyl, C₁-C₆ alkoxy, halogen, cyano, or nitro. The method includes the steps of
a) providing a compound represented by the formula (Formula II): 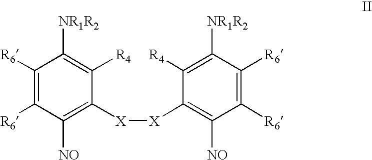
b) reacting the compound of Formula II with a compound represented by the structure (Formula III): 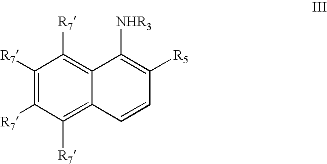
under conditions such that a compound represented by Formula Ib is formed.

In a preferred embodiment, the step of providing the compound of Formula II comprises nitrosating a compound represented by Formula V: 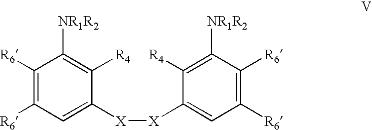
under conditions such that a compound of Formula II is provided. In further preferred embodiments, the step of nitrosating a compound represented by Formula V comprises contacting the compound of Formula V with a nitrite salt under acidic conditions. In certain preferred embodiments, the step of reacting the compound of Formula II with the compound of Formula III includes the use of trifluoroethanol (a polar weak acid).

In another embodiment, the invention provides a method for preparing a compound represented by Formula VI: 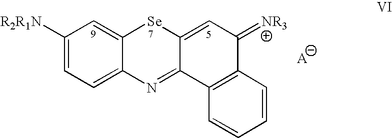
in which R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure; and A is an anion (such as F, Cl, Br, and the like). In general, the method includes the steps of:
a) contacting a compound represented by Formula VII: 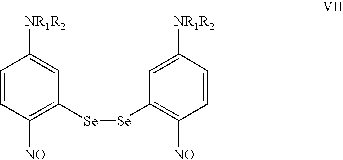
with a 1-aminonaphthalene compound under conditions such that a compound represented by Formula VIII is formed; 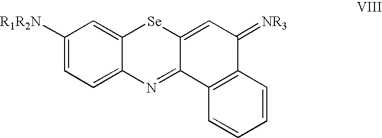
and
b) contacting the compound of Formula VIII with an acid of the form HA, in which A is an anion, under conditions such that the compound of Formula VI is formed.

In another embodiment, the invention provides a method for preparing a compound represented by Formula V: 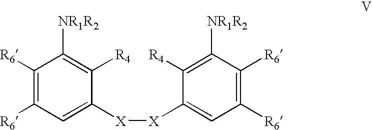
in which

R₁ and R₂ are each independently C₁-C₆ alkyl or aralkyl; or R₁ and R₂, together with the N atom to which they are attached, can form an optionally substituted heterocyclic ring having from 3 to 8 atoms in the heterocyclic ring structure;

R₄ and R₆′ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and one R₆′, together with the N atom to which R₂ is attached and two or more atoms in the ring to which R₆′ is attached, can form an optionally substituted heterocyclic ring having from 5 to 8 atoms in the heterocyclic ring structure;

the method comprising the steps of:
a) providing a compound represented by Formula IX: 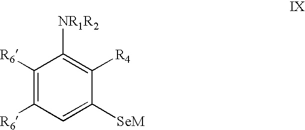
in which M is a metal cation or a proton; and
b) oxidizing the compound of Formula IX under conditions such that the compound of Formula V is formed.

In a preferred embodiment, the step of providing the compound of Formula IX comprises reacting a compound of Formula X 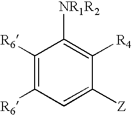
in which

R₁ and R₂ are each independently C₁-C₆ alkyl or aralkyl; or R₁ and R₂, together with the N atom to which they are attached, can form an optionally substituted heterocyclic ring having from 3 to 8 atoms in the heterocyclic ring structure;

R₄ and R₆′ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and one R₆′, together with the N atom to which R₂ is attached and two or more atoms in the ring to which R₆′ is attached, can form an optionally substituted heterocyclic ring having from 5 to 8 atoms in the heterocyclic ring structure; and Z is Cl, Br, or I; with magnesium under anhydrous conditions to form an organomagnesium intermediate; and

reacting the organomagnesium intermediate with sulfur or selenium such that the compound of Formula IX is formed.

Other aspects of the invention are described in or are obvious from the following disclosure and are within the ambit of the invention.

### BRIEF DESCRIPTION OF THE FIGURES

The following Detailed Description, given by way of example, but not intended to limit the invention to specific embodiments described, may be understood in conjunction with the accompanying figures, incorporated herein by reference. Various preferred features and embodiments of the present invention will now be described by way of non-limiting examples and with reference to the accompanying figures, in which:

FIG. 1 shows absorption and fluorescence emission spectra of selenium analog 3 in ethanol.

FIG. 2 shows a graph of survival fraction vs. fluence to depict the photodynamic inactivation of the Gram-positive E. faecalis (cell density 10⁸/mL), wherein the bacteria were incubated with 2 μM dyes, washed, and illuminated with the appropriate wavelength of red light. Values are means of three independent experiments, and bars show standard deviation (SD).

FIG. 3 shows a graph of survival fraction vs. fluence to depict the photodynamic inactivation of Gram-negative E. coli (cell density 10⁸/mL), wherein the bacteria were incubated with 5 μM dyes, washed, and illuminated with red light. Values are means of three independent experiments, and bars show SD.

FIG. 4 shows a graph of survival fraction vs. fluence to depict the photodynamic inactivation of the pathogenic yeast C. albicans (cell density 10⁸/mL) incubated with 20 μM dyes, washed, and illuminated with red light. Values are means of three independent experiments, and bars show SD.

FIG. 5 shows graphs of survival fraction vs. fluence to depict the photodynamic inactivation of E. faecalis using (a) EtNBSe (3) and (b) EtNBS (2) at 2 μM and (c) the photodynamic inactivation of E. coli using EtNBSe (3) at 5 μM, each with and without a wash. Values are means of three independent experiments, and bars show SD.

FIG. 6 shows bar graphs depicting (a) the uptake of the three photosensitizers by the three microbial species, with concentrations of 2 μM for E. faecalis, 5 μM for E. coli, and 20 μM for C. albicans, and wherein values are means of three independent experiments, and bars show SD; *, **, ***, P<0.05, 0.01, 0.001, respectively, versus EtNBA (1); ## P, <0.01 versus 1; and (b) the fraction of added photosensitizer that is bound to cells, with conditions as in (a).

### DETAILED DESCRIPTION OF THE INVENTION

### I. Definitions

In order that the invention may be more readily understood, certain terms are first defined and collected here for convenience. Other definitions appear in context throughout the application.

The term “alkyl” refers to the radical of saturated aliphatic groups, including straight-chain alkyl groups and branched-chain alkyl groups. In preferred embodiments, a straight chain or branched chain alkyl has 30 or fewer carbon atoms in its backbone (e.g., C₁-C₃₀ for straight chain, C₃-C₃₀ for branched chain), preferably 20 or fewer, and more preferably 10 or fewer, especially 6 or fewer. The term “alkyl” also includes unsaturated aliphatic groups analogous in length and possible substitution to the alkyls described above, but that contain at least one double or triple bond respectively. Moreover, the term alkyl as used throughout the specification and claims is intended to include both “unsubstituted alkyls” and “substituted alkyls,” the latter of which refers to alkyl moieties having substituents, e.g., 1-3 substitutents, replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Unsubstituted alkyl (including cycloalkyl) groups or groups substituted by halogen, especially fluorine, are, in certain embodiments, preferred over other substituted groups.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, propyl (n-propyl and i-propyl), butyl (tert-butyl, n-butyl and sec-butyl), pentyl, hexyl, heptyl, octyl and so forth. In preferred embodiment, the term “lower alkyl” includes a straight chain alkyl having 4 or fewer carbon atoms in its backbone, e.g., C₁-C₄ alkyl.

Thus specific examples of alkyl include C₁₋₆ alkyl or C₁₋₄alkyl (such as methyl or ethyl). Specific examples of hydroxyalkyl include C₁₋₆hydroxyalkyl or C₁₋₄hydroalkyl (such as hydroxymethyl).

The terms “alkenyl” and “alkynyl” refer to unsaturated aliphatic groups analogueous in length and possible substitution to the alkyls described above, but that contain at least one double or triple bond, respectively.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

An “aralkyl” moiety is an alkyl substituted with an aryl (e.g., phenylmethyl (benzyl)). The term “aryl” as used herein, refers to the radical of aryl groups, including 5- and 6-membered single-ring aromatic groups that may include from zero to four heteroatoms, for example, benzene, pyrrole, furan; thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “cycloalkyl” refers to a cyclic alkyl moiety, i.e., an alkyl moiety which forms a carbocyclic ring structure. Preferred cycloalkyls have from 3-10 carbon atoms in their ring structure, and more preferably have 3, 4, 5, 6 or 7 carbons in the ring structure. Cycloalkyls can be further substituted, e.g., with the substituents described above.

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., C₁₋₆haloalkyl or C₁₋₄haloalkyl such as fluoromethyl and trifluoromethyl.

The term “heteroatom” as used herein means an atom of any element other than carbon or hydrogen. Preferred heteroatoms are nitrogen, oxygen, sulfur and phosphorus.

In certain preferred embodiments, the term “substantially purified” refers to preparations of a compound which are substantially pure, i.e., at least 80%, 85%, 90%, 95%, 98%, or 99% pure. For example, in certain embodiments, a “substantially purified” compound is essentially free of contaminants or impurities, including any possible stereoisomers (such as diasteromers, enantiomers, or geometric isomers).

A “chalcogen analog” of benzophenoxazinium is a benzophenoxazinium having its oxygen atom substituted with a sulfur or selenium.

The term “photoactive agent” or “photodynamic agent” refers to a photoactivatable compound, or a biological precursor thereof, that produces a reactive species (e.g., oxygen) having a photochemical (e.g., cross linking) or phototoxic effect on a cell, cellular component or biomolecule upon irradiation with electromagnetic energy of the appropriate wavelength.

The term “subject” is used herein to refer to a living animal that carries an unwanted organism, the unwanted organism being the target of the therapeutic methods described herein. The subject can be a mammal, such as a human or a non-human mammal (e.g., a dog, cat, pig, cow, sheep, goat, horse, rat, or mouse). The subject may be further immune deficient; presently or previously undergoing treatment for cancer (e.g., by chemotherapy or radiation therapy); or presently or previously undergoing antibiotic therapy or an immunosuppressive therapy.

The term “obtaining” as in “obtaining the chalcogen analog” is intended to include purchasing, synthesizing or otherwise acquiring the chalcogen analog (or indicated substance or material).

As used herein, an “unwanted organism” means an organism which causes or aggravates a disorder, such as an infection, granuloma, or other adverse immune response.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless otherwise defined, all technical and scientific terms used herein have the same meaning as commonly understood by one of ordinary skill in the art to which this invention belongs. In case of conflict, the present application, including definitions will control.

### II. Methods of the Invention

Methods of the present invention comprise administration of a photoactivate chalcogen analog of a benzophenoxazinium dye for the treatment or eradication of infection in a subject.

Methods of the invention target an unwanted organism (i.e., a target organism), which is unwanted in that it infects a host organism and causes or aggravates a disease or disorder in that host.

Target organisms of the invention can be cellular. Such target organisms include at least a boundary cell membrane and are capable of energy production, nucleic acid synthesis, and contain ribosomes and are capable of ribosomal protein synthesis. Cells can be unicellular or multicellular, and the unicellular organisms can be prokaryotic or eukaryotic. The unwanted organism may be contained within a host cell, such as a phagocyte (e.g., a macrophage). Further, within that cell, the pathogen may be contained (wholly or partly) within a vacuole, vesicle, or organelle.

Eukaryotic target organisms include fungi, for example, yeasts. Candida albicans Cryptococcus neoformns, Aspergillus fumigatus, Blastomyces dermatididis, Coccidioides immitis, Pneumocystis carinii, and Histoplasma capsulatum are target pathogens contemplated herein.

Prokaryotic target organisms include bacteria. Bacteria can be Gram negative or Gram positive, which are lacking cell walls. The Gram stain basis of distinguishing bacteria, based on whether or not cells of a specific strain or species of bacteria take up a stain, or are stained with the counterstain only, is known to those of skill in the art.

Gram negative bacterial genera suitable as target organisms include Escherichia coli, Pseudomonas aeruginosa, Klebsiella pneumoniae, Proteus mirabilis, Acinetobacter baumannii, Hemophilus pneumoniae, Salmonella typhimurium and Vibrio vulnificus. Gram positive bacterial genera suitable as target organisms include Enterococcus faecalis, Staphylococcus aureus, Streptococcus pyogenes, Listeria monocytogenes and Actinomyces naeslundii.

Target organisms may also include bacteria in spore form. Bacterial spores to be inactivated can be those of any bacterial species known in the art. In one embodiment, the contaminating bacterial spores to be inactivated are those produced by bacteria of the genus Bacillus. In another embodiment, the bacterial spores to be inactivated are those produced by bacteria of the genera Clostridius, Methylosinus, Azotobacter, Bdellovibrio, Myxococcus, and Cyanobacteria. In a specific embodiment, the bacterial spores to be inactivated are Bacillus anthracis, Bacillus cereus, Bacillus thuringiensis, Bacillus subtilis or Bacillus atrophaeus species.

In specific embodiments, target organisms can be found on any light-accessible surfaces or in light-accessible areas, for example, in human and animal subjects. In the cases of humans and animals, infections of the epidermis, oral cavity, nasal cavity, sinuses, ears, lungs, urogenital tract, and gastrointestinal tract are light accessible. Epidermal infections include subcutaneous infections, especially localized lesions, which infections are light-accessible. Infections of the peritoneal cavity, such as those resulting from burst appendicitis, are light accessible via at least laparoscopic devices. A variety of skin infections which are refractory to antibiotics or long-term antifungal treatment, for example, dermatophycoses of the toenail, are suitable for photodynamic therapy using the methods of the invention.

Lung infection can occur with a variety of bacterial genera and species, which include the Pseudomonads, which are the primary cause of death of cystic fibrosis patients, Klebsiella, and can also occur with a variety of virus strains. As pathogens of the lung are increasingly resistant to classical antibiotic therapies, photodynamic therapy with the compositions of the instant invention offer an alternative method for eliminating these unwanted organisms that is independent of the microbial mechanisms of resistance. Additional epidermal infections and infections of deeper tissues arise from burns, scrapes, cuts, and puncture wounds. Photodynamic therapy with the compositions of the instant invention is useful for sterilization of such potential infectious sites, which can rapidly lead to toxic shock, a frequent concomitant of bullet wounds, and for treating the sites to eliminate or reduce unwanted infectious organisms. A major cause of infection in wounds, especially burns, is the Gram negative aerobic bacterium Pseudomonas. This organism produces an exotoxin which has been shown to retard wound healing. Multi-antibiotic resistant P. aeruginosa strains are becoming a significant problem, especially in burns units of large hospitals. Pseudomonads also produce fulminating infections of the cornea. Escherichia coli along with Staphylococcus aureus are the two most common bacteria in infected wounds.

Other sites of unwanted target organisms include the urogenital tract, the peritoneal cavity, the inner and outer ear, the nasal cavity and the gastrointestinal tract. Infectious sites of proliferation of unwanted target organisms in tissues of mesothelial and endothelial origin are also accessible to PDT by minimally invasive techniques. Additional sites of unwanted target organisms are tissues that are not well perfused or tissues wherein the organisms are present as a biofilm.

In other specific embodiments, areas of infection are not light-accessible. Such areas can be accessed, for example, with the use of light-emitting probes or catheters. Thus, delivery of the light to a recessed, or otherwise inaccessible physiological location can be facilitated by flexible fiber optics (implicit in this statement is the idea that one can irradiate either a broad field, such as the lung or a lobe of the lung, or a narrow field where bacterial or fungal cells may have localized). The source of the light needed to inactivate the bacteria or fungi can be an inexpensive diode laser or a non-coherent light source.

Other indications contemplated for treatment using the methods of the invention include surgical wound infections, acute soft tissue infections, abscesses, burn infections, chronic and acute sinusitis, periodontal disease, dental caries, bacterial keratitis and keratomycosis, otitis media, urinary tract infections, Helicobacter pylori stomach infection, dermatophytosis, mucosal candidiasis, streptococcal pharyngitis, pneumonia and cystic fibrosis lung infection, pseudomembranous colitis, prostatitis, chancroid, Buruli ulcer, mycetoma, and invasive fungal infections.

Unwanted organisms contemplated herein to be targeted for destruction include any infectious pathogens. Pathogens include, for example, viruses, bacteria, parasites, and fungi.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses). Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M avium, M intracellulare, M kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus monilifommis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

Other fungal, viral and bacterial groups and genera not listed here will be recognized by the skilled artisan as candidates for treatment by the methods of the invention. Thus, the above lists are used to illustrate applications of the present invention to major groups of suitable target organisms, but not to delimit the invention to the species, genera, families, orders or classes so listed.

Chalcogen Analogs

The photoactive agent employed in the methods of the invention is any chalcogen analog of a benzophenoxazinium dye (“chalcogen analog”). Such chalcogen analogs include, without limitation, chalcogen (O, S, Se)-substituted benzo[a]phenoxazinium analogs. IN specific embodiments, replacing the oxygen atom with sulfur and selenium significantly increases the quantum yields of single oxygen generation.

Selenium chalcogen analogs (EtNBSe) include 5-ethylamino-9-diethylaminobenzo[a]phenoselenazinium chlorides; sulfur chalcogen analogs (EtNBS) include 5-ethylamino-9-diethylaminobenzo[a]phenothiazinium chlorides; and oxygen chalcogen analogs (EtNBA) include 5-ethylamino-9-diethylaminobenzo[a]phenoxazinium chlorides.

In certain embodiments, chalcogen analogs of the invention can be represented by the following structure (Formula I): 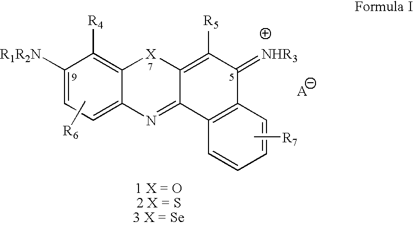
in which

X is selected from the group consisting of O, S, and Se;

R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure;

R₄ and R₅ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and R₆, together with the N atom to which R₂ is attached and two or more atoms in the ring to which R₆ is attached, can form an optionally substituted heterocyclic ring having from 5 to 8 atoms in the heterocyclic ring structure; or R₃ and R₅, together with the N atom to which R₃ is attached and two atoms in the ring to which R₅ is attached, can form an optionally substituted heterocyclic ring having from 5 to 8 atoms in the heterocyclic ring structure; or R₃ and R₇, together with the N atom to which R₃ is attached and two or more atoms in the ring system to which R₇ is attached, can form an optionally substituted heterocyclic ring having from 5 to 8 atoms in the heterocyclic ring structure;

R₆ and R₇ each independently represents 0-2 groups selected from the group consisting of C₁-C₆ alkyl, C₁-C₆ alkoxy, halogen, cyano, or nitro; and

A is an anion.

In certain preferred embodiments, in a compound of Formula I, X is S or Se. In certain preferred embodiments of Formula I, R₁, R₂ and R₃ are each independently C₁-C₆ alkyl; in more preferred embodiments, each of R₁, R₂ and R₃ is ethyl. In certain preferred embodiments of Formula I, R₄ and R₅ are each independently H, C₁-C₆ alkyl or C₁-C₆ alkoxy; in more preferred embodiments, R₄ and R₅ are each H. In certain preferred embodiments, when R₄ is a group other than H, then at least one of R₁ and R₂ is H, to decrease potential steric interactions. In certain preferred embodiments, when R₅ is a group other than H, then R₃ is H, to decrease potential steric interactions. In certain preferred embodiments, R₆ and R₇ are absent. In certain preferred embodiments, A is a monovalent or divalent anion, more preferably a monovalent anion, more preferably an anion selected from the group consisting of fluoride, chloride, bromide, iodide, tosylate (p-toluenesulfonate), mesylate (methylsulfonate), triflate (trifluoromethylsulfonate), acetate, trifluoroacetate, and benzoate.

The chalcogen analogs useful in the present invention can be prepared by a variety of methods, some of which are known in the art. For example, the synthesis of certain oxygen- and sulfur-containing chalcogen analogs is known, and the synthesis of a selenium-containing chalcogen analog is described hereinbelow.

In general, chalcogen analogs of the invention are water soluble, moderately lipophilic, and strong absorbers of red light (630-660 nm). Water solubility of the chalcogen analogs can be improved by formulation, for example, in a liposomal delivery vehicle.

In one embodiment, the invention provides a method for preparing a compound represented by Formula Ib, 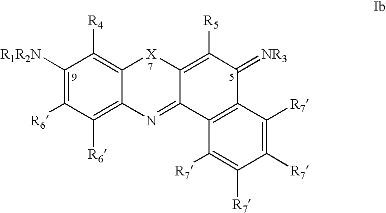
in which X is S or Se; R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure; R₄ and R₅ are each independently H, C₁-C₆ alkyl, cycloalkyl, aryl, C₁-C₆ alkoxy, halogen, cyano, or nitroor R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and R₆, together with the N atom to which R₂ is attached and two or more atoms in the ring to which R₆ is attached, can form an optionally substituted heterocyclic ring having from 5 to 8 atoms in the heterocyclic ring structure; or R₃ and R₅, together with the N atom to which R₃ is attached and two atoms in the ring to which R₅ is attached, can form an optionally substituted heterocyclic ring having from 5 to 8 atoms in the heterocyclic ring structure; or R₃ and R₇, together with the N atom to which R₃ is attached and two or more atoms in the ring system to which R₇ is attached, can form an optionally substituted heterocyclic ring having from 5 to 8 atoms in the heterocyclic ring structure; and R₆′ and R′₇ are, independently for each occurrence, H, C₁-C₆ alkyl, C₁-C₆ alkoxy, halogen, cyano, or nitro. The method includes the steps of
a) providing a compound represented by the formula (Formula II): 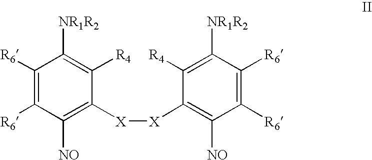
b) reacting the compound of Formula II with a compound represented by the structure (Formula III): 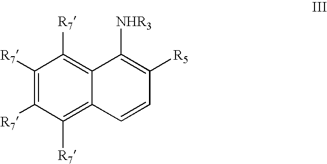
in which R₃, R₅ and R₁₇ are as described for Formula Ib, under conditions such that a compound represented by Formula Ib is formed.

In a preferred embodiment, the step of providing the compound of Formula II comprises nitrosating a compound represented by Formula V: 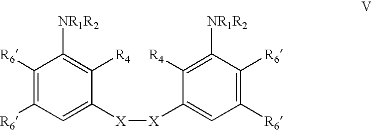
under conditions such that a compound of Formula II is provided. In further preferred embodiments, the step of nitrosating a compound represented by Formula V comprises contacting the compound of Formula V with a nitrite salt under acidic conditions. In certain embodiments, the nitrite salt is sodium nitrite. In certain embodiments, the acidic conditions comprises a solution of a mineral acid such as hydrochloric acid.

In certain preferred embodiments, the step of reacting the compound of Formula II with the compound of Formula III includes the use of a solvent comprising a polar weak acid such as trifluoroethanol. In certain embodiments, reaction may be performed in trifluoroethanol as a solvent. In certain embodiments, the reaction is performed at a temperature in the range of 30°-100° C., more preferably between about 50° C. and about 90° C. In certain embodiments, the reaction is performed at about 73-75° C. (e.g., in refluxing trifluoroethanol).

In another embodiment, the invention provides a method for preparing a compound represented by Formula V1: 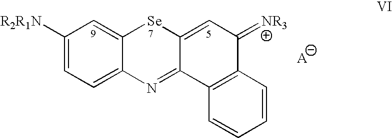
in which R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure; and A is an anion (such as F, Cl, Br, and the like). In general, the method includes the steps of:
a) contacting a compound represented by Formula VII: 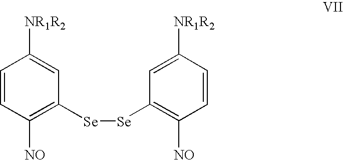
with a 1-aminonaphthalene compound under conditions such that a compound represented by Formula VIII is formed; 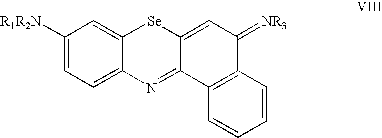
and
b) contacting the compound of Formula VIII with an acid of the form HA, in which A is an anion, under conditions such that the compound of Formula VI is formed. As used herein, the term “1-aminonaphthalene compound” refers to a naphthalene ring system or naphthalene derivative or analog having an —NH₂ group In certain embodiments, the 1-aminonaphthalene compound is represented by the formula (Formula III) as shown above.

In certain embodiments, the step of contacting the compound of Formula VII with a 1-aminonaphthalene compound includes the use of a solvent comprising a polar weak acid such as trifluoroethanol. In certain embodiments, reaction may be performed in trifluoroethanol as a solvent. In certain embodiments, the reaction is performed at a temperature in the range of 30°-100° C., more preferably between about 50° C. and about 90° C. In certain embodiments, the reaction is performed at about 73-75° C. (e.g., in refluxing trifluoroethanol).

In another embodiment, the invention provides a method for preparing a compound represented by Formula V: 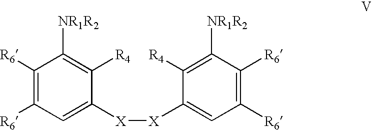
in which

R₁ and R₂ are each independently C₁-C₆ alkyl or aralkyl; or R₁ and R₂, together with the N atom to which they are attached, can form an optionally substituted heterocyclic ring having from 3 to 8 atoms in the heterocyclic ring structure;

R₄ and R₆′ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and one R₆′, together with the N atom to which R₂ is attached and two or more atoms in the ring to which R₆′ is attached, can form an optionally substituted heterocyclic ring having from 5 to 8 atoms in the heterocyclic ring structure; and

X is S or Se;

the method comprising the steps of:

a) providing a compound represented by Formula IX: 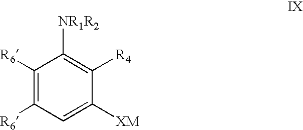
in which R₁, R₂, R₄, R′₆, and X are as described for Formula V, and M is a metal cation or a proton; and
b) oxidizing the compound of Formula IX under conditions such that the compound of Formula V is formed. In certain embodiments, the step of oxidizing comprises reacting the compound of Formula IX (e.g., in which M is a proton) with an oxidizing agent such as oxygen gas (e.g., by bubbling air through a solution of a compound of Formula IX).

In a preferred embodiment, the step of providing the compound of Formula IX comprises reacting a compound of Formula X 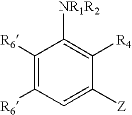
in which

R₁ and R₂ are each independently C₁-C₆ alkyl or aralkyl; or R₁ and R₂, together with the N atom to which they are attached, can form an optionally substituted heterocyclic ring having from 3 to 8 atoms in the heterocyclic ring structure;

R₄ and R₆′ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and one R₆′, together with the N atom to which R₂ is attached and two or more atoms in the ring to which R₆′ is attached, can form an optionally substituted heterocyclic ring having from 5 to 8 atoms in the heterocyclic ring structure;

and Z is Cl, Br, or I; with magnesium under anhydrous conditions, followed by reaction of the organomagnesium intermediate (Grignard reagent) with selenium or sulfur (e.g., powdered selenium or sulfur) under conditions such that the compound of Formula IX is formed. In general, the formation of the Grignard reagent is performed under anhydrous conditions in an inert solvent such as diethyl ether, and generally is performed under an inert atmosphere such as argon or nitrogen gas. The reaction with sulfur or selenium can advantageously occur by addition of the sulfinur or selenium (e.g., as a powder) to the Grignard reaction mixture, i.e., in a one-pot synthesis from the haloaromatic starting material of Formula X. In a preferred embodiment, X is Se.

The invention also includes kits for treating infections in a subject comprising a chalcogen analog and instructions for using the chalcogen analog to treat the infection in accordance with the methods described herein.

Targeting Moieties

To increase the specificity of the chalcogen analog for its target, a targeting moiety be covalently associated according to conjugation methods well known in the art.

A targeting moiety binds to a receptor, an antigenic determinant, or other binding site present on or in the target organism. Accordingly, the targeting moiety can be a molecule or a macromolecular structure that interacts with a pathogen. In one aspect of the invention, the targeting moiety can be a polypeptide (e.g., a human polypeptide such as poly-lysine or serum albumin). Alternatively, the targeting moiety can be a small anti-microbial peptide (i.e. a peptide containing less than 60 amino acid residues). Histatins, defensins, cecropins, magainins, Gram positive bacteriocins, and peptide antibiotics which meet this limitation are SAMP's. Many SAMP's are in the range of 20-40 amino acid residues in length. SAMP's are naturally occurring peptides, and are made by a wide variety of organisms. SAMP's are NPM's. Many SAMP's have a broad spectrum of antimicrobial activity, and, e.g., can kill more than one species, and in some cases can kill distantly related species, e.g. Gram negative and Gram positive bacterial species.

A targeting moiety can be directed to the infectious pathogen. In addition, certain structural features of enzymes of the organism can be targeted. Alternatively, host molecules that target the bacteria, such as anti-microbial peptides (e.g., granulysin), can be used to target chalcogen analogs of the invention (Stenger et al., Science 282:121-125, 1998).

The targeting moiety can be a polypeptide. The polypeptide may be linear, branched, or cyclic. The targeting moiety can include a polypeptide having an affinity for a polysaccharide target, for example, a lectin (such as a seed, bean, root, bark, seaweed, fungal, bacterial, or invertebrate lectin). Particularly useful lectins include concanavalin A, which is obtained from jack beans, and lectins obtained from the lentil, Lens culinaris.

Desirable characteristics for the targeting moieties include: specificity for one or more unwanted target organisms or components thereof (e.g. cell surface receptors), affinity and avidity for such organisms, and stability with respect to conditions of coupling reactions and the physiology of the organ or tissue of use. Specificity need not be narrowly defined, e.g., it may be desirable for a targeting molecule to have affinity for a broad range of target organisms, such as all Gram negative bacteria. The targeting moiety, when incorporated into a composition to be provided in a method of the invention, should be nontoxic to the cells of the subject.

Targeting moieties can be selected from the sequences of naturally occurring proteins and peptides, from variants of these peptides, and from biologically or chemically synthesized peptides. Naturally occurring peptides which have affinity for one or more target organism can provide sequences from which additional peptides with desired properties, e.g., increased affinity or specificity, can be synthesized individually or as members of a library of related peptides. Such peptides can be selected on the basis of affinity for the target organism.

Naturally occurring peptides with affinity for target organisms useful in methods and compounds of the invention, include aptomers, salivary proteins, e.g., histatins, microbially-elaborated proteins, e.g., bacteriocins, peptides that bind and/or kill species that are closely related to the producing strains; and proteins produced by animal species such as defensins, which are produced by mammals, and the cecropins and magainins, produced by moths and amphibia, respectively.

As mentioned briefly above, histatins, defensins, cecropins and magainins are examples of a class of polypeptides found widely in nature, which share the characteristics of small size (generally approximately 30 amino acid residues, and between 10 residues and 50 residues), broad specificity of anti-microbial activity, and low affinity for target organisms.

Histatins are a family of histidine-rich cationic polypeptides which have bactericidal and candidacidal properties and are constituents of normal human saliva (Oppenheim, G. G. et al., J. Biol. chem. 263:7472-747, 1988). Their mechanism of action is thought to involve a combination of alpha-helical conformation and cationic charge leading them to insert between the polar head groups in the bacterial cell wall (Raj, P. A. et al., J. Biol. Chem. 269:9610-9619, 1994).

Bacteriocins, which are proteins produced by bacteria and which kill other strains and species of bacteria (Jack, R. W. et al., Microbiol. Rev. 59:171-200, 1995) can be used as targeting moieties. An exemplary Gram positive bacteriocin is nisin, produced by Lactococcus lactis and accorded GRAS status (generally regarded as safe) by the Food and Drug Administration for application to food preservation.

The bacteriocins nisin, subtilin, epidermin, gallidermin, salivarin, and lacticin exemplify the “lantibiotic” class of Gram positive bacteriocin, which is defined as a bacteriocin in which one or more cysteine residues are linked to a dehydrated serine or threonine to form a thioether-linked residue known as lanthionine (Lan) or threo-.beta.-methyllanthionine (MeLan). These are post-translational modifications found in these anti-microbial peptides by the producing cell. Lantibiotics contain leader peptide sequences of 18-24 residues, which are cleaved to yield an active antimicrobial peptide of about 22-35 residues. Growth of the producing bacterial species, and preparation and purification of bacteriocins are performed by published procedures and techniques which can be carried out by one of skill in the art. For example, Yang, R. et al., Appl. and Env. Microbiol. 58: 3355-3359, 1992, describe purification of bacteriocins from each of 4 genera of lactic acid bacteria, by optimizing absorption onto the producing cells, followed by use of low pH for selective elution of greatly enriched bacteriocin fractions. Mutant forms of each of the bacteriocins nisin, produced by Lactococcus lactis, and of subtilin, produced by Bacillus subtilis have more desirable properties than the parental wild-type forms (Liu, W. and N. Hansen, J. Biol. Chem. 267:25,078-25,085, 1992). Procedures for isolation of appropriate genes and for mutagenesis and selection of strains carrying desirable mutations are found in Maniatis, T. et al, 1982, Molecular Cloning: a Laboratory Manual, Cold Spring Harbor Press, Cold Spring Harbor, N.Y., and in the subsequent second edition, Sambrook, J. et al., 1989.

Anti-microbial peptides are produced by a variety of animals (Saberwal, G. and R. Nagaraj, Biochim. Biophys. Act. 1197:109-131, 1994). An example is a peptide of the cecropin family produced by Cecropia moths. Several cecropins contain 37 residues, of which 6 are lysine. Cecropins are active against both Gram positive and Gram negative bacteria. Other insect-produced peptides include apidaecin (from honeybees), andropin (from fruit flies), and cecropin family members from bumble bees, fruit flies, and other insects.

The defensins are produced by mammals, including humans, and are generally about 29-34 residues in length, and the magainins (about 23 residues) are produced by amphibia such as Xenopus laevis. Defensins from human (HNP-1,-2,-3 and 4), guinea pig (GPNP), rabbit (NP-1, -2, -3A, -3B, -4 and -5) and rat (NP-1, -2, -3 and -4) share a significant number of regions of homology. Defensins can have antimicrobial activity against Gram positive bacteria or Gram negative bacteria and fungi, with minimal inhibitory concentrations in the mM range. Rabbit NP-1 and NP-2 are more potent antibacterial agents than others in this family. Other mammalian anti-microbial peptides include murine cryptdin, bovine granulocyte bactenecin and indolicidin, and seminal-plasmin from bovine semen. Additional amphibial anti-microbials include PGLA, XPF, LPF, CPG, PGQ, bombinin from Bombina variegata, the bombinin-like peptides BLP-1, -2, -3 and -4 from B. orientalis, and brevinins from Rana esculenta. Invertebrates such as the horseshoe crab can be a source of anti-microbial peptides such as the tachyplesins (I, II and III) and the polyphemusins (I and II).

Peptides in these families of antimicrobial agents are generally cationic, and can have a broad antimicrobial spectrum, including both antibacterial and antifungal activities. The addition of positively charged residues can enhance antimicrobial specific activity several fold. The positive charges are thought to assist in the insertion of the peptides into the membranes of the susceptible organisms, in which context the peptide molecules can form pores and cause efflux of ions and other metabolites. Structural studies of the Moses sole fish neurotoxin 33 residue peptide pardaxin, for example, reveals that succinylated pardaxin inserts into erythrocyte and model membranes more slowly than unmodified pardaxin. (Shai, Y et al., J. Biol. Chem. 265: 20, 202-20, 209, 1990). The positively charged magainin molecule can disrupt both the metabolism of E. coli and the electric potential of the mitochondrion (Westerhoff, H. V., et al., Proc. Natl. Acad. Sci. 86:6597-6601, 1989).

Novel peptides, for example, a cecropin-melittin hybrid, and synthetic Denantiomers have antimicrobial activity (Merrifield, R. B. et al., “Antimicrobial peptides,” Ciba Foundation Symp. 186, John Wiley, Chichester, pp. 5-26, 1994). One such synthetic cecropin-melittin peptide is 5-fold more active against Mycobacterium smegmatis than rifampin.

Targeting moieties can be plant proteins with affinities for particular target organisms, for example, a member of the lectin protein family with affinity for polysaccharides. Targeting moieties can be synthetic peptides, such as polylysine, polyarginine, polyornithine, and synthetic heteropolypeptides that comprise substantial proportions of such positively charged amino acid residues. Such peptides can be chemically synthesized or produced biologically in recombinant organisms, in which case the targeting moiety peptide can be produced as part of a larger protein, for example as the N-terminus residues, and cleaved from that larger protein. Polypeptides suitable as “backbone” moieties are also suitable as target moieties, if they have sufficient affinity for the target organism. Considerations described are thus appropriate to consideration of a targeting moieties.

Targeting moieties need not be limited to peptide compositions, but can be lectins, polysaccharides, steroids, and metalloorganic compositions. Targeting moieties can be comprised of compositions that are composed both of amino acids and sugars, such as mucopolysaccharides. A useful targeting moiety can be partially lipid and partially peptide in nature, such as low density lipoprotein. Serum lipoproteins especially high density and low density lipoproteins (HDL and LDL) can bind to bacterial surface proteins (Emancipator, K. et al., Infect. Immun. 60:596-601, 1992). The appropriate binding features of the lipoproteins to bacterial surface components can be identified by methods of molecular biology known in the art, and the binding feature of lipoproteins can be used as the targeting moiety in chalcogen analogs of the present invention.

Administration of Chalcogen Analogs

The chalcogen analogs described herein can be provided to a subject in a free form, i.e., in solution. Alternatively the compositions can be delivered in various formulations including, but not limited to, liposome, peptide-bound, polymer-bound, or detergent-containing formulations. Those of ordinary skill in the art are well able to generate and administer such formulations. The composition should be soluble under physiological conditions, in aqueous solvents containing appropriate carriers or excipients, or in other systems, such as liposomes, that may be used to administer the conjugate to a subject. Chalcogen analogs that are somewhat insoluble in an aqueous solvent can be applied in a liposome, or a time release fashion, such that illumination can be applied intermittently using a regimen of periods of illumination alternating with periods of non-illumination. Other regimens contemplated are continuous periods of lower level illumination, for which a time-release formulation is suitable.

A composition as described herein can be administered by a variety of methods known in the art, including orally and topically. In one aspect, the chalcogen analog may be administered parenterally. The phrase “administered parenterally” as used herein means modes of administration other than oral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

As will be appreciated by the skilled artisan, the route and/or mode of administration will vary depending upon the desired results. A chalcogen analog as described herein can be contained in a pharmaceutically acceptable excipient or carrier. Included, without limitation, are any and all solvents, dispersion media, coatings, antibacterial and antifungal agents, isotonic and absorption delaying agents, and the like that are physiologically compatible. The use of such media and agents for pharmaceutically active substances is well known in the art. Preferably, the carrier is suitable for oral, intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

In one aspect, the carrier may protect the compound against rapid release, for example, a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In another aspect of the invention, the chalcogen analogs can be administered by combination therapy, i.e., combined with other agents. For example, the combination therapy can include a composition as described herein with at least one other anti-microbial (e.g., antibiotic), or other conventional therapy. The combination therapy could also include, for example, co-administration of the chalcogen analog together with an inhibitor of multi-drug resistance (e.g., an MDR inhibitor).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

One of ordinary skill in the art can determine and prescribe the effective amount of the chalcogen analog as required. For example, one could start doses of the known or novel chalcogen analog levels lower than that required in order to achieve the desired therapeutic effect and gradually increase the dosage until the desired effect is achieved. For example, the dosage may range from 0.1 mg/kg to 10 mg/kg depending on the therapeutic agent used.

The iterations delineated above are not intended as limiting with respect to the nature of the compositions described herein, or to a particular route of the administration.

Photoactivation of Chalcogen Analogs

Typically, administration of a chalcogen analog as described herein is followed by a sufficient period of time to allow accumulation thereof at the target site. The chalcogen analog can, subsequently, be activated by irradiation. This is accomplished by applying light of a suitable wavelength and intensity, for an effective length of time, at the site of the inflammation. As used herein, “irradiation” refers to the use of light to induce a chemical reaction of a photoactive chalcogen analog.

Photoactivating dosages depend on various factors, including the amount of the photosensitizer administered, the wavelength of the photoactivating light, the intensity of the photoactivating light, and the duration of illumination by the photoactivating light. Thus, the dose can be adjusted to a therapeutically effective dose by adjusting one or more of these factors. Such adjustments are within the level of ordinary skill in the art. Irradiation of the appropriate wavelength for a given compound may be administered by a variety of methods. Methods for irradiation include, but are not limited to, the administration of laser, nonlaser, or broad band light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments that provide transillumination. Delivery of the light to a recessed, or otherwise inaccessible physiological location can be facilitated by flexible fiber optics (implicit in this statement is the idea that one can irradiate either a broad field, such as the lung or a lobe of the lung, or a narrow field where bacterial cells may have localized). The source of the light needed to inactivate the bacteria can be an inexpensive diode laser or a non-coherent light source.

The chalcogen analogs described herein should be stable during the course of at least a single round of treatment by continued or pulsed irradiation, during which the chalcogen analog within the composition would, preferably, be repeatedly excited to the energized state, undergoing multiple rounds of generation of singlet oxygen.

The suitable wavelength, or range of wavelengths, will depend on the particular photosensitizer(s) used, and can range from about 350 nm to about 550 nm, from about 550 nm to about 650 nm, from about 650 nm to about 750 nm, from about 750 nm to about 850 nm and from about 850 nm to about 950 nm.

In specific embodiments, target tissues are illuminated with red light. Given that red and/or near infrared light best penetrates mammalian tissues, photosensitizers with strong absorbances in the range of about 600 nm to about 900 nm are optimal for photodynamic therapy. For photoactivation, the wavelength of light is matched to the electronic absorption spectrum of the photoactive agent so that the photoactive agent absorbs photons and the desired photochemistry can occur. Wavelength specificity for photoactivation generally depends on the molecular structure of the photoactive agent. Photoactivation can also occur with sub-ablative light doses. Determination of suitable wavelength, light intensity, and duration of illumination is within ordinary skill in the art.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the photoactive agent can alter, these values. In general, photoactive agents with longer absorbing wavelengths and higher molar absorption coefficients at these wavelengths are more effective photodynamic agents.

The light for photoactivation can be produced and delivered to the site of inflammation by any suitable means known in the art. Photoactivating light can be delivered to the site of inflammation from a light source, such as a laser or optical fiber. Preferably, optical fiber devices that directly illuminate the site of inflammation deliver the photoactivating light. For example, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Optical fibers can also be passed through arthroscopes. In addition, light can be transmitted by percutaneous instrumentation using optical fibers or cannulated waveguides. For open surgical sites, suitable light sources include broadband conventional light sources, broad arrays of light-emitting diodes (LEDs), and defocused laser beams.

Delivery can be by all methods known in the art, including transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

Where treatment is desired, the dosage of chalcogen analog, and light activating the chalcogen analog, is administered in an amount sufficient to produce a phototoxic species. There is a reciprocal relationship between chalcogen analogs and light dose, thus, determination of suitable wavelength, light intensity, and duration of illumination is within ordinary skill in the art.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. One of ordinary skill in the art can determine and prescribe the effective amount of the pharmaceutical composition required. For example, one could start doses of the known or novel chalcogen analog levels lower than that required in order to achieve the desired therapeutic effect and gradually increase the dosage until the desired effect is achieved. Irradiation of the appropriate wavelength for a given compound may be administered by a variety of wavelengths. Methods for irradiation include, but are not limited to, the administration of laser, nonlaser, or broad band light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments that provide transillumination.

The wavelength and power of light can be adjusted according to standard methods known in the art to control the production of phototoxic species. Thus, under certain conditions (e.g., low power, low fluence rate, shorter wavelength of light or some combination thereof), a fluorescent species is primarily produced from the photoactive agent and any reactive species produced has a negligible effect. These conditions are easily adapted to bring about the production of a phototoxic species. Determination of suitable wavelength, light intensity, and duration of illumination for chalcogen analog is within the level of ordinary skill in the art.

Kits

In another aspect, the invention provides a kit for decreasing the activity of an unwanted organism in a subject. In preferred embodiments, the kit includes a chalcogen analog of a benzophenoxazinium dye and instructions for using the chalcogen analog for decreasing the activity of an unwanted organism in a subject.

In preferred embodiments, the kit comprises a sterile container which contains the chalcogen analog; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments.

The instructions will generally include information about the use of the compound for decreasing the activity of an unwanted organism in a subject; in preferred embodiments, the instructions include at least one of the following: description of the chalcogen analog compound; dosage schedule and administration for treatment of a disease or disorder or symptoms thereof associated with an unwanted organism in a subject, or instructions for decreasing the activity of an unwanted organism in a subject; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The present invention is additionally described by way of the following illustrative, non-limiting Examples that provide a better understanding of the present invention and of its many advantages.

### EXAMPLES

Described herein are the broad-spectrum antimicrobial sensitizing efficacies of a series of three chalcogen (O, S, Se)-substituted benzo[a]phenoxazinium analogs against a panel of prototypical human pathogenic microbes consisting of the Gram-positive bacterium Enterococcus faecalis, the Gram-negative bacterium Escherichia coli, and the yeast Candida albicans.

### Example 1

### Preparation of Chemical Compounds

General Chemical Synthesis Information

All solvents were reagent grade and used as received. All intermediates and dyes were purified using medium pressure (100 psi) chromatography using Woelm 32-63 silica gel. Thin layer chromatography was performed using commercially prepared silica gel covered glass plates (Whatman K5F 150A). UV/visible absorption spectra were recorded using an HP 8453 spectrophotometer. Corrected fluorescence spectra were recorded using a SPEX Fluorolog2 (solvent measurements) or a FluoroMax3 (microorganism measurements), SPEX Industries, Edison, N.J. Fluorescence quantum yields were measured relative to Cresyl Violet standard at 25° C.³² Quantum yields for ¹O₂ formation were determined by measuring the ¹O₂-mediated bleaching of 1,3-diphenylisobenzofuran¹² relative to Methylene Blue (0.50)³³ at 25° C. ¹H NMR spectra were obtained using a Varian 400 MHz spectrometer with TMS as internal standard. Chemical shifts are reported in δ, coupling constants as J (cps) using standard peak splitting terminology. HPMS (electrospray ionization (ESI)) were obtained from the Mass Spectroscopy Facility, Chemical and Chemical Biology Department, Harvard University.

5-Ethylamino-9-diethylaminobenzo[a]phenoxazinium (1, EtNBA) and 5-ethylamino-9-diethylaminobenzo[a]phenothiazinium (2, EtNBS) were available from earlier studies and had been prepared using previously described procedures.²⁹⁻³¹

### Bis-(3-N,N-diethylaminophenyl) diselenide (5)

To magnesium turnings (1.94 g, 80 mmol) stirred in fresh, dry ethyl ether (50 mL) under an argon atmosphere was added dropwise over a 3 hour period an ethyl ether (50 mL) solution of 3-iodo-N,N-diethylaniline³⁴ (4) (10.0 g, 36.4 mmol) and 1,2-dibromoethane (6.84 g, 36.4 mmol). After an additional hour of stirring most of the magnesium had reacted. To the resulting Grignard reagent was slowly added, via a flexible solids addition tube, selenium powder (3.35 g, 42.4 mmol, 325 mesh); the addition rate (15 min) was modulated so as to keep a controlled, gentle solvent reflux which results from the exotherm of the reaction. The resulting mixture was stirred overnight at room temperature. The reaction was carefully quenched with the dropwise addition of 30 mL water (initial vigorous reaction) to give a dark orange ether layer and a brown water layer. Air was bubbled through this stirred mixture overnight; the exhaust, which contained selenium vapor (TOXIC), was trapped by bubbling through a solution of aqueous sodium hypochlorite (bleach). The residue was taken up in ethyl ether (250 mL) and washed with water, brine and was dried over sodium sulfate. Removal of the solvent in vacuo afforded 7.35 g of a yellow oil that was purified using silica gel chromatography using 1% ethyl ether in hexane as eluent to afford 4.61 g (56%) of diselenide 5 as a light yellow oil. TLC analysis indicated that the product contained a trace of a single impurity that was below the detection limits of NMR spectroscopy; 5 was used without further purification. ¹H NMR (δ/ppm, CDCl₃): 1.10 (t, J=7.2, 12H), 3.28 (q, J=7.2, 8H), 6.51 (dd, J=7.6, 2.0, 2H), 6.90 (m, 4H), 7.06 (dd, J=8.4, 7.6, 2H). HRMS (ESI) m/z C₂₀H₂₈N₂Se₂ (MH+) calcd, 457.0661, found, 457.0655.

### Bis-(3-N,N-diethylamino-6-nitrosophenyl) diselenide (6)

To a stirred, cold (5° C.) solution of diselenide 5 (2.36 g, 5.2 mmol) in 1N hydrochloric acid (200 mL) was added an aqueous solution sodium nitrite (0.72 g, 11.4 mmol), whereupon the clear yellow solution rapidly formed an orange precipitate. After an additional 10 minutes of stirring, the mixture was extracted twice with methylene chloride (200 mL). The organic layer was washed twice with brine and dried over sodium sulfate. Solvent was removed in vacuo to give 2.22 g of an orange/brown solid. Crystallization from 2-propanol gave 1.84 g (69%) of brown crystals. TLC analysis (5% methanol in methylene chloride) showed the desired product contained a trace amount of a blue contaminant that could not be removed by an additional crystallization. Thus, 6 was used without further purification. ¹H NMR (6/ppm, CDCl₃): 1.43 (broad m, 12H), 3.70 (broad m, 8H), 7.13 (dd, J=9.6, 2.4, 2H), 7.84 (d, J=2.4, 4H), 8.38 (d, J=9.6, 2H). HRMS (ESI) m/z C₂₀H₂₆N₄O₂Se₂ (MH+) calcd, 515.0464, found, 515.0458. 5-Ethylamino-9-diethylaminobenzo[a]phenoselenazinium chloride (3, EtNBSe) A stirred solution of bis-nitroso diselenide 6 (1.70 g, 3.3 mmol) and 1-N-ethylnaphthylamine, 7, (1.49 g, 9.6 mmol) in trifluoroethanol was heated to reflux temperature. Initially, the reaction solution showed the formation of an absorption band at 810 nm that was rapidly replaced by a deep blue band at 660 nm. Reaction was complete in less than 1 h, whereupon solvent was removed in vacuo to give a blue waxy solid. The solid was stirred twice with ethyl ether to remove excess 7 and dissolved in a mixture of aqueous 1N sodium hydroxide and methylene chloride. The color of the solution turned bright magenta indicating that deprotonation of 3 had occurred. The mixture was placed in a separatory funnel, and the organic layer was washed twice with brine. Dye 3 was regenerated as a deep blue chloride salt by adding 0.5 mL of concentrated hydrochloric acid to the magenta methylene chloride solution. After removing solvent and excess hydrochloric acid in vacuo, the desired product was purified by column chromatography using a solvent gradient of methanol (2-7% v/v) in methylene chloride. Fractions that consisted of a single spot by TLC analysis were combined to give 3 (1.49 g, 55%) as a non-crystalline solid. Because 3 binds tenaciously to a trace amount of solvent (NMR analysis) which we were unable to remove using high vacuum and heat, the dye was twice dissolved in anhydrous ethanol and evaporated to dryness in order to exchange residual eluting solvent with this innocuous alcohol. ¹H NMR (6/ppm, MeOH-d₄): 1.32 (t, J=6.8, 6H, N(CH₂CH₃)₂), 1.44 (t, J=7.2, 3H, NH(CH₂CH₃)), 3.65 (two overlapping q, J=7.2 and 6.8, 6H, both types of N—CH₂), 7.13 (dd, J=9.6, 2.4, 1H), 7.40 (d, J=2.8, 1H), 7.55 (s, 1H), 7.74 (m, 1H), 7.82 (m, 1H), 7.96 (d, J=9.6, 1H), 8.21 (d, J=8.4 1H), 8.99 (d, J=8.4, 1H). HRMS (ESI) m/z C₂₂H₂₄N₃Se (M+) calcd, 410.1135, found, 410.1138.

Combustion elemental analysis for compound 3

| C₂₂H₂₄N₃SeCl-0.5 H₂O | % C | % H | % N |
|---|---|---|---|
| Calculated | 58.22 | 5.55 | 9.26 |
| Found | 58.28 | 5.54 | 9.27 |

Of note, the literature contains no prior example of a benzo[a]phenoselenazinum dye and only a single reference to an unsymmetrical phenoselenazinium dye which had been synthesized by Groves, et. al. via the acid-catalyzed condensation of a p-nitroso-N,N-dialkylaniline with bis-(3-aminophenyl) diselenide.³⁷ Although initial efforts for preparing EtNBSe (3) using an identical sequence of reactions as those used in this latter work were unsuccessful, a modification of the Groves approach, as outlined in Scheme 1, below, did afford the agent in high yield. 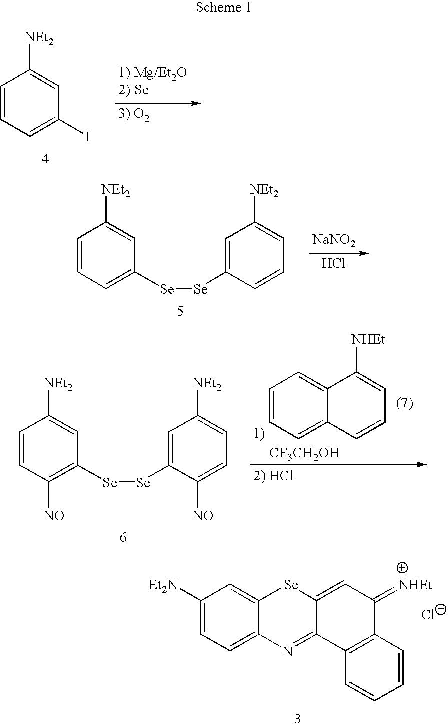
The Grignard reagent derived from 3-iodo-N,N-diethylaniline, 4, was converted to diselenide 5 by reaction with selenium powder followed by air oxidation. Treatment of 5 with two equivalents of nitrous acid gave dinitroso diselenide 6 as an orange solid. Attempts to condense 6 with N-ethyl-1-naphthylamine, 7, in ethanol gave no reaction; increasingly better yields of EtNBSe (3) were realized in this solvent as progressively weaker acids, including HCl, acetic acid and acetic acid-sodium acetate, were used as the catalyst. This trend naturally led us to evaluate trifluoroethanol, which is mildly acidic and non-nucleophilic, as both solvent and benign catalyst whereupon the desired dye was formed cleanly and in good yield. Treatment with an ion exchange procedure ensured that chloride was the counter ion of 3; subsequent column chromatography afforded the chloride salt of EtNBSe (3) as a dark blue solid (55% yield).

### Example 2

### Physical and Photophysical Properties of Chalcogen Chloride Dyes

Inspection of the molecular structures of three selected chalcogen chloride dyes described herein, as presented below, shows that they are relatively small, planar and possess a delocalized positive charge that can be neutralized by the removal of the proton from the C-5 amino group. 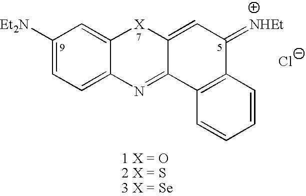
The distinguishing feature of each is the nature of the chalcogen atom residing at the 7 ring position. The generic molecular structure of the series is closely related to that of Nile Blue A, a commercial stain, differing by an additional ethyl moiety on the 5-amino group. The inclusion of this substituent was discovered to greatly increase aqueous solubility while concomitantly increasing lipophilicity. This may be attributed to added steric bulk that accompanies incorporation of the additional alkyl group which decreases the tendency of the dyes to form large, less water soluble aggregates.
Photostability of Photosensitizers

In order to ensure that the photosensitizers described herein, especially selenium derivative 3, are resistant to photodegradation by singlet oxygen, a water-cooled 1 cm² square cuvette containing 3 mL of a methanol/acetic acid; (100:1, v/v) solution of each dye (OD=1.0) was placed in a cell holder. One face of the cuvette was subjected to the unfiltered beam emanating from a slide projector (Polaroid 610, fluence 72 Jcm⁻² at an irradiance of 20 mWcm⁻² for 600-700 nm band). The optical density (OD) and the wavelength of maximum absorption of each solution were compared before and after illumination. In all cases, the OD of the solutions decreased less than 4%, while the wavelengths of maximum absorption, a sensitive probe for detecting changes at the 7 chalcogen-substituted ring position³⁵, remained unchanged, indicating that, at least in the relatively benign environment afforded by ethanol, all of the dyes are relatively stable. Physical and photophysical data relevant to the studies described herein are presented in Table 1, below.

TABLE 1
Physical and photophysical properties of pertinent dyes.

| Dye | λ_{abs}(nm)^{a} | λ_{fl}(nm)^{b} | Φ_{fl}^{c} | ¹O₂^{d} | LogP^{e} |
|---|---|---|---|---|---|
| 1 | 632 | 660 | 0.27 | 0.003 | 2.69 |
| 2 | 654 | 693 | 0.21 | 0.03 | 2.76 |
| 3 | 661 | 703 | 0.03 | 0.78 | 2.08 |

^{a}Absorption maximum in ethanol containing 0.1% acetic acid.
^{b}Fluorescence maximum in ethanol.
^{c}Absolute fluorescence quantum yield.
^{d}Absolute quantum yield for singlet oxygen formation.
^{e}Partition coefficient between 2-octanol and pH 7.4 buffered saline.

All three dyes are efficient absorbers of red light having extinction coefficients greater than 50,000 L/mol-cm ¹⁸. As expected, the wavelength of maximum absorption shifts to longer wavelengths as the chalcogen atom is varied from oxygen to sulfur and selenium.³⁸ Absorption data is presented for the dyes dissolved in ethanol rather than water because even at very low concentrations in aqueous media all three chromophores form, not only monomeric species, but also H-dimers and possibly higher order aggregates, which we deduce from the appearance of an additional broad absorption band in the short red spectral region (data not shown); in ethanol the dyes appear to be completely monomeric as evidenced by their adherence to Beer's Law and the absence of the short red band.

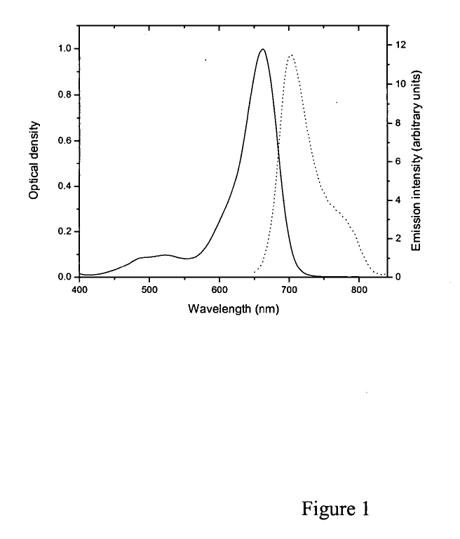

All three chalcogen chromophores fluoresce, having wavelengths of maximum emission shifted approximately 40 nm to the red of their respective absorbance maxima. This relationship is illustrated in FIG. 1, which provides an example of the absorption and emission curves for selenium dye 3; the other two members of the series have similarly shaped spectral profiles.

The relative photoactivities of the three photosensitizers studied herein were gauged by their abilities to generate singlet oxygen (¹O₂) when illuminated with a beam of red light having an adjusted intensity such that each dye absorbed the same number of photons per unit time. As shown in Table 1, above, the quantum yields of ¹O₂ formation, measured using the 1,3-diphenylisobenzofuran method, rose dramatically as the atomic number of the chalcogen atom, and, hence, the spin-orbital coupling constants, increased from O to Se. Finally, because of the relevance of lipophilicity to the present studies, the partition coefficients for the PS between 2-octanol and phosphate-buffered saline (pH=7.4) are listed¹⁸.

### Example 3

### Antimicrobial Studies

Microbial Strains

The microorganisms studied were Escherichia coli (ATCC 25922), Enterococcus faecalis (ATCC 29212), and Candida albicans (ATCC 18804). The microorganism species chosen to evaluate the photoactivities of the dyes in the present study are important pathogens in the type of infections, such as wounds and burns, likely to be amenable to photodynamic therapy (PDT). E. faecalis is typical of Enterococci that frequently develop vancomycin resistance and are important nocosomial pathogens in post-surgical wounds and burns.⁴² Although E. coli is generally thought of as an intestinal or urinary tract pathogen, there is increasing concern about its presence in wound infections and its ability to develop extended spectrum beta-lactamase resistance.^{43,44} C. albicans is increasingly found in nocosomial infections in burns⁴⁵ and, to a lesser extent, in surgical wounds.⁴⁶

Cells were grown at 37° C. in aerobic conditions in a shaker at 150 rpm. Brain-heart infusion broth (Difco, BD Diagnostic Systems, Sparks, Md.) was used for E. coli and E. faecalis, YM medium (Difco) was used for C. albicans. Exponential cultures obtained by reculturing stationary overnight precultures were used for all experiments. E. coli and E. faecalis were grown in fresh medium for approximately 1 h to a density of 10⁸ cells/mL; the OD values at 650 nm were 0.6. C. albicans was grown for approximately 4 h to approximate density of 10⁷ cells/mL corresponding to an OD of 6 at 650 nm (ten fold dilution measured). Exact cell numbers were confirmed by counting colony forming units obtained after serial dilution on square BHI (or YM for Candida) agar plates.

Photosensitizer solutions and light sources

Stock solutions (2 mM) of the three chalcogen dyes were prepared in water and stored at 4° C. in the dark for no longer than 1 month before use. A non-coherent light source with interchangeable fiber bundles (LumaCare, London, UK) was employed. Thirty-nm band pass filters allowed total powers of roughly 1 W to be obtained from 630+/−15 nm for EtNBA (1), 652+/−15 nm for EtNBS (2), and 660+/−15 nm for EtNBSe (3).

Dye Uptake by Microbial Cells

Suspensions of microorganisms were incubated for 10 minutes with photosensitizer in the dark at room temperature. Unbound photosensitizer was washed out by centrifugation of the mixture of dye and microorganisms for 6 minutes at 1550g, followed by resuspension of washed pellets in 10 mL PBS without Ca⁺⁺/Mg⁺⁺. Aliquots (200 μL) of these suspensions were used for photodynamic inactivation (PDI) experiments; the remaining suspensions were centrifuged again, and the pellets were dissolved in 3 mL 10% SDS for at least 24 hours. These suspensions were used for photosensitizer uptake measurement. The fluorescence of dissolved pellets was measured on a spectrofluorimeter (FluoroMax3, SPEX Industries, Edison, N.J.). For EtNBA (1), the excitation wavelength was 625 nm, and the emission spectra of the solution were recorded from 630 to 750 nm. For EtNBS (2), the excitation wavelength was 645 nm, and the range for emission was 650 to 750nm. For EtNBSe (3), the excitation wavelength was 655 nm, and the emission was recorded in the range from 660 to 750 nm. The fluorescence was calculated from the height of the peaks recorded. Calibration curves were made from pure photosensitizer dissolved in 10% SDS and used for determination of sensitizer concentration in the suspension. Uptake values were obtained by dividing the number of nmol of sensitizer in the dissolved pellet by the number of colony-forming units (CFU) obtained by serial dilutions and the number of photosensitizer molecules/cell calculated using Avogadro's number.

Photodynamic Inactivation Studies

Illumination was performed either after or before excess dye was washed out. Aliquots of 200 μL of cell suspension were placed in 96-well plates and illuminated with appropriate light at room temperature. Fluences ranged from 0 to 80 Jcm⁻² at an irradiance of 60-100 mWcm⁻². Exact power was measured with a laser power meter (model FM/GS, Coherent, Santa Clara, Calif.). During illumination after defined fluences had been delivered, aliquots of 20 μL were taken to determine the colony-forming units (CFU). The contents of the wells were mixed before sampling. The aliquots were serially diluted 10-fold in PBS without Ca⁺⁺/Mg⁺⁺ to give dilutions of 10⁻¹-10⁻⁶ times the original concentrations and were streaked horizontally on square BHI agar plates, as described by Jett, et al.³⁶ Plates were incubated at 37° C. overnight. Colonies were counted and survival fraction determined compared to untreated control. Dye in the absence of light and light alone were also used as controls. Photosensitizers were generally nontoxic for microorganisms in the dark, and light alone did not cause cell destruction. All experiments were performed in triplicate.

Statistics

Values are reported as means +/−SD. Differences between two means were evaluated by the unpaired, two-sided Students t -test assuming equal or unequal variation in the standard deviations as appropriate. Differences between the slopes of killing curves were evaluated using linear regression analysis function contained in GraphPad Prism software (GraphPad Software Inc, San Diego, Calif.). P values of less than 0.05 were considered significant.

Photodynamic inactivation of E. faecalis

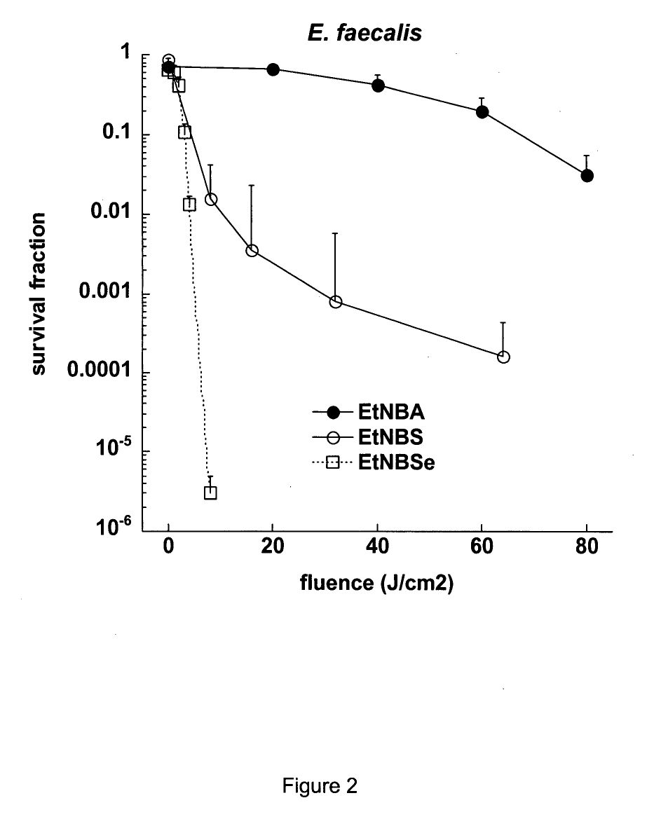

The Gram-positive bacterium E. faecalis was killed by all three photosensitizers at a concentration of 2 μm followed by a wash and application of red light, but there were large and significant differences between the degrees of killing observed (FIG. 2). Because preliminary experiments showed that the dyes had very different activities, more light was used to kill cells incubated with EtNBS (2, up to 64 J/cm²) and EtNBA (1, up to 80 j/cm²) than was used for EtNBSe (3, up to 32 J/cm²). EtNBSe (3) was by far the most effective photosensitizer, leading to killing of >99.999% of cells (5.5 logs, P<0.001) after the delivery of only 8J/cm² of 660-nm light. EtNBS (2) was the next most efficient antimicrobial photosensitizer, with killing of >99.9% (3 logs) after delivery of 64 J/cm² of 652-nm light, while EtNBA (1) was the least effective photosensitizer, requiring the delivery of 80 J/cm² 6f 635-nm light to the kill of 95% of the cells.

Photodynamic inactivation of E. coli

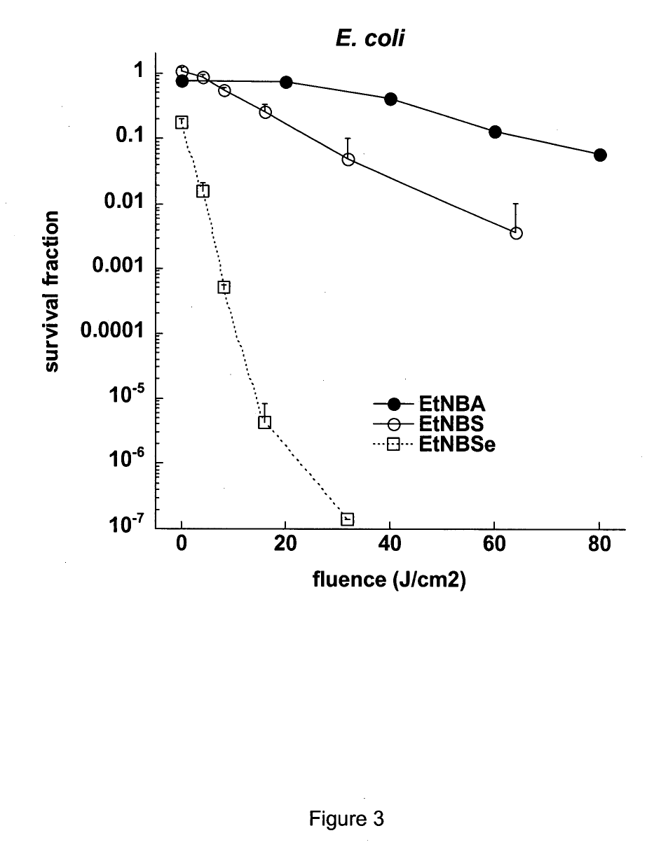

A somewhat higher concentration (5 μM) of photosensitizer was used to test the PDT killing of the Gram-negative E. coli after a wash, because Gram-negative species are generally found to be more resistant to PDT than Gram-positive species.²⁰ Again, EtNBSe (3) was by far the most effective photosensitizer, with delivery of 32 J/cm² giving almost total eradication of >99.9999% (>6 logs, P<0.001) (FIG. 3). EtNBS (2) was much less effective, leading to killing of slightly more than 99% after 64 J/cm², while EtNBA (1) was again the least effective, only killing 90% of cells after 80 J/cm².

Photodynamic inactivation of C. albicans

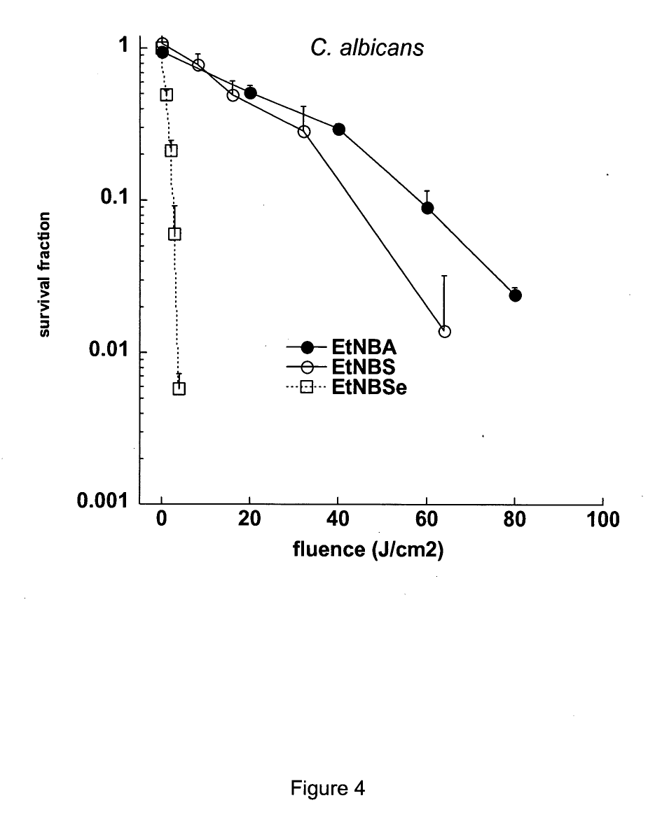

Because Candida cells are ten times larger than bacterial cells²², an even higher photosensitizer concentration was employed than that used for bacterial cells, i.e., 20 μM with a wash. Under these conditions, EtNBSe (3) was again the most effective photosensitizer by a large margin, with only 4 J/cm² required to kill more than 99% of cells (P<0.001) (FIG. 4). EtNBS (2) was less effective, needing a fluence of 64 J/cm² to give the same degree of cell killing (>98%), while EtNBA (1) was the least effective, requiring 80 J/cm² to kill >97% cells.

Effect of microorganism wash pre-PDT

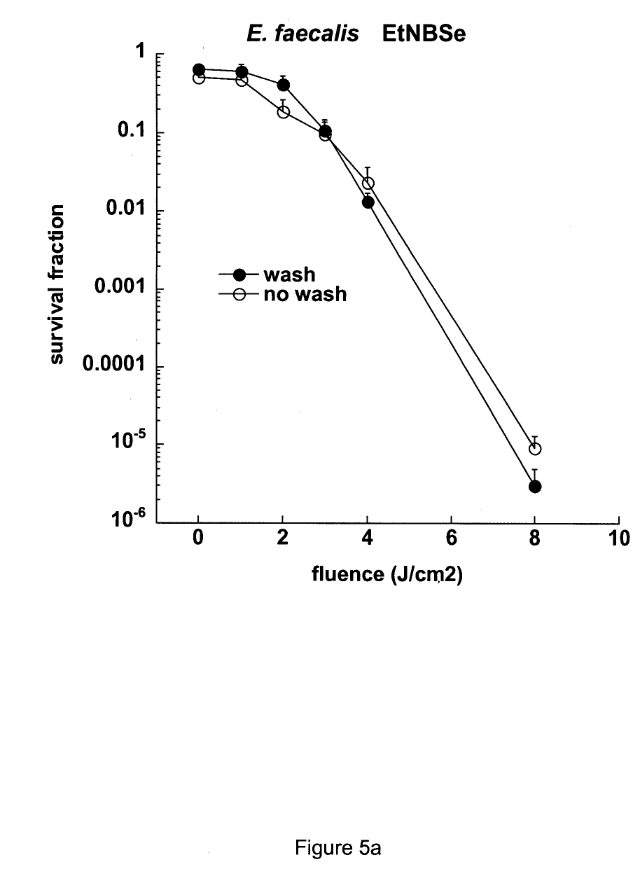

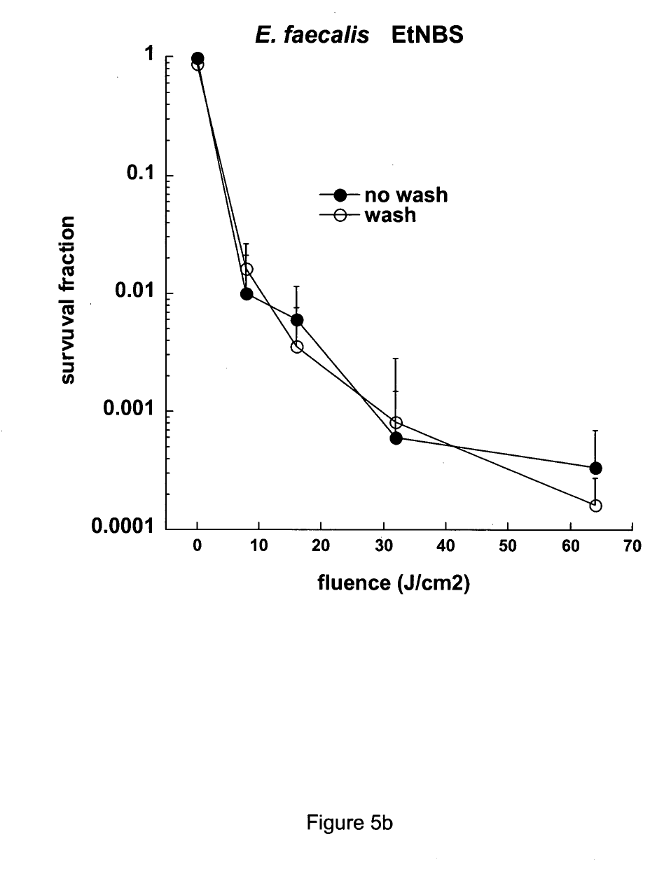

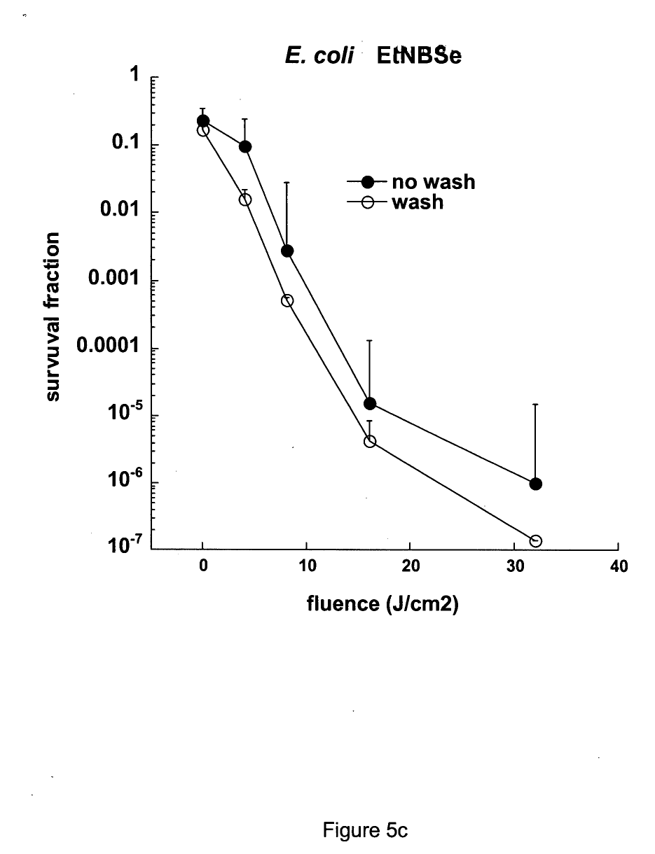

In order to test whether the photosensitizers were more or less effective at killing microbial cells when present in solution during illumination, three sets of experiments were compared with and without a wash of the unbound photosensitizer from the cell suspension. FIG. 5a shows the comparison of the killing curves for E. faecalis with 2 μM EtNBSe (3) with and without a wash. There is no significant difference in the two killing curves. FIG. 5b shows the same experiment repeated with E. faecalis with 2 μM EtNBS (2). Again the two curves are almost identical. FIG. 5c shows the same set of experiments carried out with E. coli and 5 μM of EtNBSe (3). In this case, bacteria washed free of incubating solution were actually killed slightly (but not significantly) more than those of the non-wash group.

### Example 4

### Microbial Uptake

It was also examined whether the observed large differences in effectiveness of antimicrobial photodynamic therapy of the three dyes depended on photochemical efficiencies, the degree of cell-dye binding, or both. In order to do so, the uptake of dye was measured in terms of molecules per cell by carrying out 10 minute incubations with the same concentration of dyes that was used for photoinactivation, followed by a wash to remove residual incubating solution.

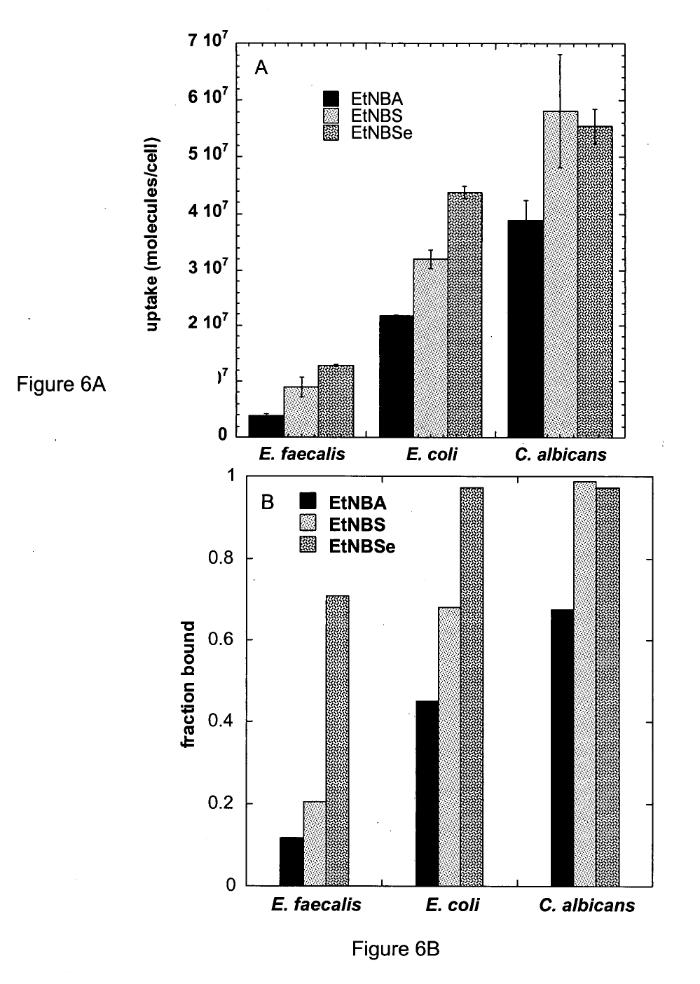

The bacterial pellets resulting from the incubation with photosensitizer were dissolved in 10% SDS to give a homogeneous solution, and comparison of the resulting fluorescence intensities with a calibration curve prepared with known concentrations of dye in the same solvent yielded the amount of dye in the pellet. FIG. 6A shows that for E. faecalis, the cell uptake was in the order EtNBSe (3)>EtNBS (2)>EtNBA (1). The differences between EtNBA (1) and both EtNBS (2) and EtNBSe (3) wer& significant, but the difference between EtNBS (2) and EtNBSe (3) was not significant. A similar order of cell uptake (but higher values due to higher dye concentrations being used) was found for E. coli, with EtNBSe (3)>EtNBS (2)>EtNBA (1). In this case, the differences between all three values were significant. For the fungus C. albicans, an even higher concentration of dye (20 μM) was used, because the eukaryotic cells are ten times bigger than the prokaryotic bacterial cells. The uptake of EtNBA (1) was significantly lower than that found for EtNBSe (3).

By comparing the amount of dye that remained in solution with the dye that was in the bacterial pellet, it was possible to calculate the fraction of total dye added that was bound to the cells, as shown in FIG. 6B. The binding of EtNBSe (3) to C. albicans and E. coli, and the binding of EtNBS (2) to C. albicans, was essentially quantitative, while approximately 75% of the dye was cell-bound in the case of EtNBA (1) to C. albicans, EtNBS (2) to E. coli, and EtNBSe (3) to E. faecalis. Thus, the affinity of dyes for cells increased in the order EtNBA (1)<EtNBS (2)<EtNBSe (3), and the affinity of cells for dyes increased in the order E. faecalis <E. coli <C. albicans.

It is, thus, shown herein that a series of chalcogen-substituted phenoxazinium derivatives originally developed as anti-cancer photodynamic therapy agents are also effective broad-spectrum antibacterial agents. The physical and photophysical attributes that characterize the series and make these dyes well suited for this purpose include that: (1) they are strong absorbers of red light; (2) when illuminated with red photons, they become, or, in reaction with molecular oxygen, they generate, a cytotoxin; (3) they are lipophilic and bear a delocalized positive charge; (4) they are relatively stable to photodegradation when illuminated in alcohol at moderate light fluences; and (5) they emit red or near infrared fluorescence. Additionally, their solubility in water is of benefit for drug administration purposes, and two members of the series, 1 and 2, evaluated in prior extensive animal model studies, appear to have little or no toxicity in the dark.

The ability for an antimicrobial photosensitizer to absorb light in the 600-900-nm region of the spectrum is critically important for in vivo applications, because these are the wavelengths that penetrate tissues most effectively. All three chalcogen analogs investigated herein meet this criterion, with the oxygen-, sulfur-, and selenium-substituted photosensitizers having absorption maxima at 634 nm, 653 nm and 661 nm, respectively.

Since most photodynamic therapy agents emit a fluorescent signal upon excitation with visible light, they offer the valuable benefit of doubling as real-time in situ luminescent reporters of the location and concentration of the drug. Such is the case with the photosensitizers of the present investigation: 1 and 2 are strong emitters that can easily be detected with a fluorescence microscope, while 3 produces relatively low fluorescence quantum yield. Nevertheless, even selenium analog 3 emits with an intensity that can easily be detected by modern electronic imaging instrumentation of the type used in the quantitative studies described herein.

### REFERENCES

1. Dougherty, T. J., Photosensitizers and Therapy and Detection of Malignant-Tumors. Photochemistry and Photobiology 1987, 45, (6), 879-889.
2. Castano, A. P.; Demidova, T. N.; Hamblin, M. R., Mechanisms in photodynamic therapy: part one—photosensitizers, photochemistry and cellular localization. Photodiagnosis and Photodynamic Therapy 2004, 1, (4), 279-293.
3. Castano, A. P.; Demidova, T. N.; Hamblin, M. R., Mechanisms in photodynamic therapy: part two-cellular signalling, cell metabolism and modes of cell death. Photodiagnosis and Photodynamic therapy 2005, 2, 1-23.
4. Castano, A. P.; Demidova, .T. N.; Hamblin, M. R., Mechanisms in photodynamic therapy: part three:—photosensitizer pharmpocokinetics, biodistribution, tumor localization and modes of tumor destruction. Photodiagnosis and Photodynamic Therapy 2005, 2, 91-106.
5. Detty, M. R.; Gibson, S. L.; Wagner, S. J., Current clinical and preclinical photosensitizers for use in photodynamic therapy. Journal of Medicinal Chemistry 2004, 47, (16), 3897-3915.
6. Michl, J.; Bonacic-Koutecky, V., Electronic Aspects of Organic Photochemistry. first ed.; John Wiley & Sons, Inc: 1990.
7. Moan, J.; Peng, Q., An outline of the hundred-year history of PDT. Anticancer Research 2003, 23, (5A), 3591-3600.
8. Dolmans, D.; Fukumura, D.; Jain, R. K., Photodynamic therapy for cancer. Nature Reviews Cancer 2003, 3, (5), 380-387.
9. Lin, C.-W., Photodynamic therapy of malignant tumors: recent developments. Cancer Cells (Cold Spring Harbor) 1992, 3, 437-444.
10. Henderson, B. W.; Dougherty, T. J., How Does Photodynamic Therapy Work. Photochemistry and Photobiology 1992, 55, (1), 145-157.
11. Lin, C. W.; Shulok, J. R.; Wong, Y. K.; Schanbacher, C. F.; Cincotta, L.; Foley, J. W., Photosensitization, Uptake, and Retention of Phenoxazine Nile Blue Derivatives in Human Bladder-Carcinoma Cells. Cancer Research 1991, 51, (4), 1109-1116.
12. Cincotta, L.; Foley, J. W.; Cincotta, A. H., Novel Red Absorbing Benzo[a]Phenoxazinium and Benzo[a]Phenothiazinium Photosensitizers-Invitro Evaluation. Photochemistry and Photobiology 1987, 46, (5), 751-758.
13. Lewis, M. R.; Sloviter, H. A.; Goland, P. P., Invivo Staining and Retardation of Growth of Sarcomata in Mice. Anatomical Record 1946, 95, (2), 89-96.
14. Lewis, M. R.; Goland, P. P.; Sloviter, H. A., The Action of Oxazine Dyes on Tumors in Mice. Cancer Research 1949, 9, (12), 736-740.
15. Cincotta, A. H.; Cincotta, L.; Foley, J. W., Novel benzophenothiazinium photosensitizers: preliminay in vivo results. SPIE 1990, 1203, 202-210.
16. Cincotta, L.; Foley, J. W.; Maceachern, T.; Lampros, E.; Cincotta, A. H., Novel Photodynamic Effects of a Benzophenothiazine on 2 Different Murine Sarcomas. Cancer Research 1994, 54, (5), 1249-1258.
17. Frimberger, A. E.; Moore, A. S.; Cincotta, L.; Cotter, S. M.; Foley, J. R., Photodynamic therapy of naturally occurring tumors in animals using a novel benzophenothiazine photosensitizer. Clinical Cancer Research 1998, 4, (9), 2207-2218.
18. Cincotta, L.; Foley, J. W.; Cincotta, A. H., Phototoxicity, Redox Behavior, and Pharmacokinetics of Benzophenoxazine Analogs in Emt-6 Murine Sarcoma-Cells. Cancer Research 1993, 53, (11), 2571-2580.
19. Hamblin, M. R.; Hasan, T., Photodynamic therapy: a new antimicrobial approach to infectious disease? Photochemical & Photobiological Sciences 2004, 3, (5), 436-450.
20. Malik, Z.; Ladan, H.; Nitzan, Y., Photodynamic Inactivation of Gram-Negative Bacteria—Problems and Possible Solutions. Journal of Photochemistry and Photobiology B-Biology 1992, 14, (3), 262-266.
21. Nitzan, Y.; Gutterman, M.; Malik, Z.; Ehrenberg, B., Inactivation of Gram-Negative Bacteria by Photosensitized Porphyrins. Photochemistry and Photobiology 1992, 55, (1), 89-96.
22. Demidova, T. N.; Hamblin, M. R., Effect of cell-photo sensitizer binding and cell density on microbial photoinactivation. Antimicrobial Agents and Chemotherapy 2005, 49, (6), 2329-2335.
23. Merchat, M.; Bertolini, G.; Giacomini, P.; Villanueva, A.; Jori, G., Meso-substituted cationic porphyrins as efficient photosensitizers of gram-positive and gram-negative bacteria. Journal of Photochemistry and Photobiology B-Biology 1996, 32, (3), 153-157.
24. Voet, D.; Voet, J. G., Biochemistry. first ed.; John Wiley a& Sons, Inc.: 1990.
25. Hamouda, T.; Shih, A. Y.; Baker, J. R., A rapid staining technique for the detection of the initiation of germination of bactyerial spores. Letters in Applied Microbiology 2002, 34, 86-90.
26. Phoenix, D. A.; Harris, F., Phenothiazinium-based photosensitizers: antibacterials of the future? Trends in Molecular Medicine 2003, 9, (7), 283-285.
   27. http://www.ondinebiopharma.com/periodontal_disease_therapy.php., 2005.
28. http://www.denfotex.com/. 2005.
29. Foley, J. W.; Cincotta, L. Photo-inactivation of cancer cells. U.S. Pat. No. 4,962,197, 1990.
30. Nikas, D.C.; Foley, J. W.; Black, P. M., Fluorescent imaging in a glioma model in vivo. Lasers in Surgery and Medicine 2001, 29, (1), 11-17.
31. Clapp, R. C.; English, J. P.; Fellows, C. E.; Forsythe, J.; Grotz, R. E.; Shepherd, R. G., Chemotherapeutic Dyes 0.5. Benzo[a]Phenothiazines and Benzo[a]Phenazines. Journal of the American Chemical Society 1952, 74, (8), 1994-1996.
32. Magde, D.; Brannon, J. H.; Cremers, T. L.; Olmsted, J., Absolute Luminescence Yield of Cresyl Violet—Standard for the Red. Journal of Physical Chemistry 1979, 83, (6), 696-699.
33. Tanielian, C.; Wolff, C.; Esch, M., Singlet oxygen production in water: Aggregation and charge-transfer effects. Journal of Physical Chemistry 1996, 100, (16), 6555-6560.
34. Gregory, P.; Thorp, D., Electronic Absorption-Spectra of 2-Substituted-4-Nn-Diethylamino-4′-Nitroazobenzene Dyes and Their Monoacid Cations—Applicability of Dewar Rules to These and Related Dyestuffs. Journal of the Chemical Society-Perkin Transactions 1 1979, (8), 1990-2000.
35. Griffiths, J., Colour and Constitution of Organic Molecules. first ed.; Acedemic Press, Inc: 1976; p 264.
36. Jett, B. D.; Hatter, K. L.; Huycke, M. M.; Gilmore, M. S., Simplified agar plate method for quantifying viable bacteria. Biotechniques 1997, 23, (4), 648-650.
37. Groves, J. T.; Lindenau.Sm; Haywood, B. J.; Knol, J. A.; Schultz, J. S., Synthesis of Seleno-Toluidine Blue. Journal of Medicinal Chemistry 1974, 17, (8), 902-904.
38. Detty, M. R.; Merkel, P. B., Chalcogenapyrylium Dyes as Potential Photochemotherapeutic Agents—Solution Studies of Heavy-Atom Effects on Triplet Yields, Quantum Efficiencies of Singlet Oxygen Generation, Rates of Reaction with Singlet Oxygen, and Emission Quantum Yields. Journal of the American Chemical Society 1990, 112, (10), 3845-3855.
39. Henderson, B. W.; Miller, A. C., Effects of Scavengers of Reactive Oxygen and Radical Species on Cell-Survival Following Photodynamic Treatment Invitro—Comparison to Ionizing-Radiation. Radiation Research 1986, 108, (2), 196-205.
40. Lien, E. J.; Hansch, C.; Anderson, S. M., Structure-Activity Correlations for Antibacterial Agents on Gram-Positive and Gram-Negative Cells. Journal of Medicinal Chemistry 1968, 11, (3), 430-&.
41. Cincotta, L.; Foley, J. W.; Cincotta, A. H., Novel phenothiazinuium photosensitizers for photodynamic therapy. SPIE 1988, 997, 145-153.
42. Broder, K. W.; Moise, P. A.; Schultz, R. O.; Forrest, A.; Schentag, J. J., Clinical experience with linezolid in conjunction with wound coverage techniques for skin and soft-tissue infections and postoperative osteomyelitis. Annals of Plastic Surgery 2004, 52, (4), 385-390.
43. Russo, T. A.; Johnson, J. R., Medical and economic impact of extraintestinal infections due to Escherichia coli: focus on an increasingly important endemic problem. Microbes and Infection 2003, 5, (5), 449-456.
44. Shinagawa, N.; Hirata, K.; Katsuramaki, T.; Hata, F.; Ushijima, Y.; Ushida, T.; Tanimura, H.; Taniguchi, K.; Aikawa, N.; Yo, K.; Yamaue, H.; Ohnishi, H.; Takayama, T.; Sato, T.; Kato, K.; Tanaka, N.; Iwagaki, H.; Yura, J.; Fuchimoto, S.; Manabe, T.; Takeyama, H.; Kimura, H.; Hasegawa, M.; Takeuchi, H.; Yasui, Y.; Taniguchi, M.; Sueda, T.; Takesue, Y.; Mashita, K.; Hiyama, E.; Ishikawa, S.; Yokoyama, T.; Mizuno, A.; Ikeda, S.; Yasunami, Y.; Kubo, S.; Takada, N.; Suzuki, Y.; Fujimoto, M., Bacteria isolated from surgical infections and its susceptibilities to antimicrobial agents—special references to bacteria isolated between April 2003 and March 2004. Japanese Journal of Antibiotics 2005, 58, 123-158.
45. Still, J. M.; Law, E. J.; Belcher, K. E.; Spencer, S. A., A Comparison of Susceptibility to 5 Antifungal Agents of Yeast Cultures from Burn Patients. Burns 1995, 21, (3), 167-170.
46. Chen, Y. C.; Chang, S. C.; Sun, C. C.; Yang, L. S.; Hsieh, W. C.; Luh, K. T., Secular trends in the epidemiology of nosocomial fungal infections at a teaching hospital in Taiwan, 1981 to 1993. Infection Control and Hospital Epidemiology 1997, 18, (5), 369-375.

1. A method for decreasing the activity of an unwanted organism in a subject, said method comprising the steps of:
i) providing an effective amount of a photoactive chalcogen analog of a benzophenoxazinium dye to a pathogen; and
ii) light-activating the chalcogen analog to produce a phototoxic species, thereby decreasing the activity of the unwanted organism in the subject.

2. The method of claim 1, wherein the chalcogen analog is selected from the group consisting of a selenium chalcogen analog, sulfur chalcogen analog, and oxygen chalcogen analog.

3. The method of claim 1, wherein the selenium chalcogen analog is 5-ethylamino-9-diethylaminobenzo[a]phenoselenazinium chloride.

4. The method of claim 1, wherein the sulfur chalcogen analog is 5-ethylamino-9-diethylaminobenzo[a]phenothiazinium chloride.

5. The method of claim 1, wherein the oxygen chalcogen analog is 5-ethylamino-9-diethylaminobenzo[a]phenoxazinium chloride.

6. The method of claim 1, wherein the subject is a mammal.

7. The method of claim 4, wherein the mammal is a human.

8. The method of claim 1, wherein the unwanted organism is selected from the group consisting of a virus, bacteria and fungi.

9. The method of claim 1, wherein the unwanted organism is a fungus.

10. The method of claim 9, wherein the fungus is Candida albicans.

11. The method of claim 1, wherein the an unwanted organism is a bacteria.

12. The method of claim 11, wherein the bacteria is a Gram (+) bacteria.

13. The method of claim 12, wherein the bacteria is Enterococcus faecalis.

14. The method of claim 11, wherein the bacteria is a Gram (−) bacteria.

15. The method of claim 14, wherein the bacteria is Escherichia coli.

16. The method of claim 1, wherein the chalcogen analog further comprises a targeting moiety.

17. The method of claim 1, wherein the unwanted organism is killed.

18. The method of claim 1, further comprising the step of obtaining the chalcogen analog.

19. A kit for decreasing the activity of an unwanted organism in a subject, the kit comprising a chalcogen analog of a benzophenoxazinium dye and instructions for using the chalcogen analog in accordance with the method of claim 1.

20. The method of claim 2, wherein the chalcogen analog is represented by the structure (Formula I): 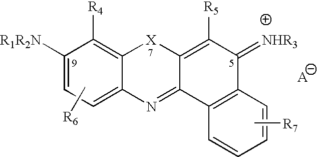
in which
X is selected from the group consisting of O, S, and Se;
R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure;
R₄ and R₅ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and R₆, together with the N atom to which R₂ is attached and two or more atoms in the ring to which R₆ is attached, can form an optionally substituted heterocyclic ring having from 5 to 8 atoms in the heterocyclic ring structure; or R₃ and R₅, together with the N atom to which R₃ is attached and two atoms in the ring to which R₅ is attached, can form an optionally substituted heterocyclic ring having from 5 to 8 atoms in the heterocyclic ring structure; or R₃ and R₇, together with the N atom to which R₃ is attached and two or more atoms in the ring system to which R₇ is attached, can form an optionally substituted heterocyclic ring having from 5 to 8 atoms in the heterocyclic ring structure;
R₆ and R₇ each independently represents 0-2 groups selected from the group consisting of C₁-C₆ alkyl, C₁-C₆ alkoxy, halogen, cyano, or nitro; and
A is an anion.

21. The method of claim 20, wherein X is S or Se.

22. The method of claim 20, wherein R₁, R₂ and R₃ are each independently C₁-C₆ alkyl.

23. The method of claim 22, in which each of R₁, R₂ and R₃ is ethyl.

24. A method for preparing a compound represented by Formula Ib, 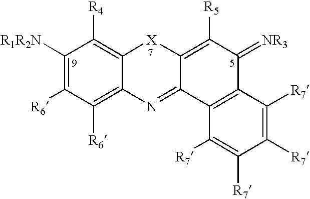
in which
X is S or Se;
R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure;
R₄ and R₅ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and R₆, together with the N atom to which R₂ is attached and two or more atoms in the ring to which R₆ is attached, can form an optionally substituted heterocyclic ring having from 5 to 8 atoms in the heterocyclic ring structure; or R₃ and R₅, together with the N atom to which R₃ is attached and two atoms in the ring to which R₅ is attached, can form an optionally substituted heterocyclic ring having from 5 to 8 atoms in the heterocyclic ring structure; or R₃ and R₇, together with the N atom to which R₃ is attached and two or more atoms in the ring system to which R₇ is attached, can form an optionally substituted heterocyclic ring having from 5 to 8 atoms in the heterocyclic ring structure; and
R₆′ and R′₇ are, independently for each occurrence, H, C₁-C₆ alkyl, C₁-C₆ alkoxy, halogen, cyano, or nitro;
the method comprising:
a) providing a compound represented by the formula (Formula II): 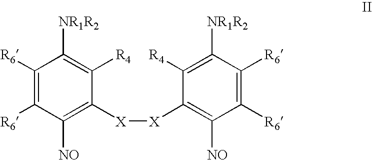
b) reacting the compound of Formula II with a compound represented by the structure (Formula III): 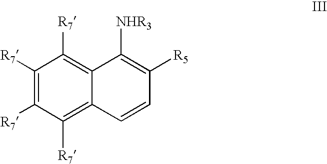
under conditions such that a compound represented by Formula Ib is formed.

25. The method of claim 24, wherein the step of providing the compound of Formula II comprises nitrosating a compound represented by Formula V: 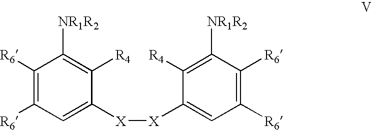
under conditions such that a compound of Formula II is provided.

26. The method of claim 25, wherein the step of nitrosating a compound represented by Formula V comprises contacting the compound of Formula V with a nitrite salt under acidic conditions.

27. A method for preparing a compound represented by Formula V1: 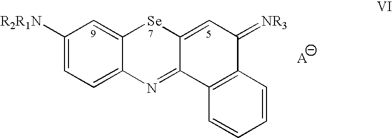
in which
R₁, R₂ and R₃ are each independently C₁-C₆ alkyl or aralkyl; or R₁ and R₂, together with the N atom to which they are attached, can form an optionally substituted heterocyclic ring having from 3 to 8 atoms in the heterocyclic ring structure; and
A is an anion;
the method comprising the steps of:
a) contacting a compound represented by Formula VII: 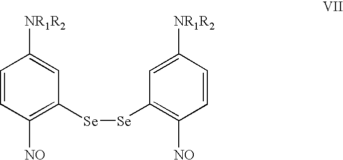
with a 1-aminonaphthalene compound under conditions such that a compound represented by Formula VIII is formed; 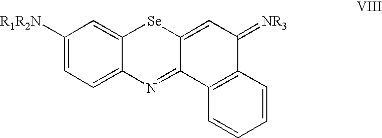
and
b) contacting the compound of Formula VIII with an acid of the form HA, in which A is an anion , under conditions such that the compound of Formula VI is formed.

28. A method for preparing a compound represented by Formula V: 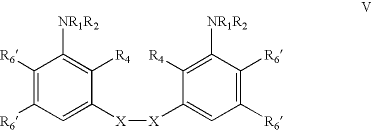
in which
R₁ and R₂ are each independently C₁-C₆ alkyl or aralkyl; or R₁ and R₂, together with the N atom to which they are attached, can form an optionally substituted heterocyclic ring having from 3 to 8 atoms in the heterocyclic ring structure;
R₄ and R₆′ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and one R₆′, together with the N atom to which R₂ is attached and two or more atoms in the ring to which R₆′ is attached, can form an optionally substituted heterocyclic ring having from 5 to 8 atoms in the heterocyclic ring structure; and
X is S or Se
the method comprising the steps of:
a) providing a compound represented by Formula IX: 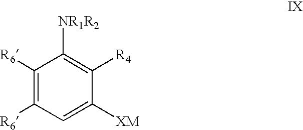
in which M is a metal cation or a proton;
b) oxidizing the compound of Formula IX under conditions such that the compound of Formula V is formed.

29. The method of claim 28, wherein the step of providing the compound of Formula IX comprises reacting a compound of Formula X 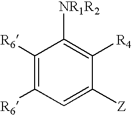
in which
R₁ and R₂ are each independently C₁-C₆ alkyl or aralkyl; or R₁ and R₂, together with the N atom to which they are attached, can form an optionally substituted heterocyclic ring having from 3 to 8 atoms in the heterocyclic ring structure;
R₄ and R₆′ are each independently H, C₁-C₆ alkyl, cycloalkyl, aryl, C₁-C₆ alkoxy, halogen, cyano, or nitro; or R₁ and R₄, together with the N atom to which R₁ is attached and two atoms in the ring to which R₄ is attached, can form an optionally substituted heterocyclic ring having from 5 to 8 atoms in the heterocyclic ring structure; or R₂ and one R₆′, together with the N atom to which R₂ is attached and two or more atoms in the ring to which R₆′ is attached, can form an optionally substituted heterocyclic ring having from 5 to 8 atoms in the heterocyclic ring structure;
and Z is Cl, Br, or I;
with magnesium under anhydrous conditions to form an organomagnesium intermediate; and
reacting the organomagnesium intermediate with sulfur or selenium such that the compound of Formula IX is formed.
